(19) United States
(12) Patent Application Publication
(10) Pub. No.: US 2002/0197648 A1
(43) Pub. Date: Dec. 26, 2002

(54) High throughput screening methods using magnetic resonance imaging agents

(76) Inventors: Robin M. Silva, Burlingame, CA (US); Thomas J. Meade, Willmette, IL (US); Douglas A. Bakan, San Diego, CA (US)

Correspondence Address: Robin M. Silva, Esq., FLEHR HOHBACH TEST ALBRITTON & HERBERT LLP, Suite 3400, Four Embarcadero Center, San Francisco, CA, 94111-4187, US

(21) Appl. No.: 10/139,145

(22) Filed: May 2, 2002

Related U.S. Application Data
(60) Provisional application No. 60/288,963, filed on May 2, 2001.

(51) Int. Cl. C12Q 1/68; G01N 33/53; A61K 49/00; C07K 14/00; C07F 5/00

(52) U.S. Cl. 435/7.1; 435/6; 424/9.34; 424/9.35; 424/9.36; 530/350; 534/15

(57) ABSTRACT

The invention relates to a wide variety of different methods and compositions that find use in high throughput screening applications utilizing magnetic resonance imaging (MRI) contrast agents.

This application This application claims the benefit of provisional application Ser. No. 60/288,963, filed May 2, 2001.

### FIELD OF THE INVENTION

The invention relates to a wide variety of different methods and compositions that find use in high throughput screening applications utilizing magnetic resonance imaging (MRI) contrast agents.

### BACKGROUND OF THE INVENTION

Magnetic resonance imaging (MRI) is a diagnostic and research procedure that uses high magnetic fields and radio-frequency signals to produce images. The most abundant molecular species in biological tissues is water. It is the quantum mechanical “spin” of the water proton nuclei that ultimately gives rise to the signal in all imaging experiments. In MRI the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans.

The Image.

MR images are typically displayed on a gray scale with black the lowest and white the highest measured intensity (I). This measured intensity I=C*M, where C is the concentration of spins (in this case, water concentration) and M is a measure of the magnetization present at time of the measurement. Although variations in water concentration (C) can give rise to contrast in MR images, it is the strong dependence of the rate of change of M on local environment that is the source of image intensity variation in MRI. Two characteristic relaxation times, T₁ & T₂, govern the rate at which the magnetization can be accurately measured. T₁ is the exponential time constant for the spins to decay back to equilibrium after being perturbed by the RF pulse. In order to increase the signal-to-noise ratio (SNR) a typical MR imaging scan (RF & gradient pulse sequence and data acquisition) is repeated at a constant rate for a predetermined number of times and the data averaged. The signal amplitude recorded for any given scan is proportional to the number of spins that have decayed back to equilibrium since the previous scan. Thus, regions with rapidly decaying spins (i.e. short T₁ values) will recover all of their signal amplitude between successive scans.

The measured intensities in the final image will accurately reflect the spin density (i.e. water content). Regions with long T₁ values compared to the time between scans will progressively lose signal until a steady state condition is reached and will appear as darker regions in the final image. Changes in T₂ (spin-spin relaxation time) result in changes in the signal linewidth (shorter T₂ values) yielding larger linewidths. In extreme situations the linewidth can be so large that the signal is indistinguishable from background noise. In clinical imaging, water relaxation characteristics vary from tissue to tissue, providing the contrast which allows the discrimination of tissue types. Moreover, the MRI experiment can be setup so that regions of the sample with short T₁ values and/or long T₂ values are preferentially enhanced so called T₁ -weighted and T₂-weighted imaging protocol.

MRI Contrast Agents.

There is a rapidly growing body of literature demonstrating the clinical effectiveness of paramagnetic contrast agents (currently 8 are in clinical trials or in use). The capacity to differentiate regions/tissues that may be magnetically similar but histologically distinct is a major impetus for the preparation of these agents [1, 2]. In the design of MRI agents, strict attention must be given to a variety of properties that will ultimately effect the physiological outcome apart from the ability to provide contrast enhancement [3]. Two fundamental properties that must be considered are biocompatability and proton relaxation enhancement. Biocompatability is influenced by several factors including toxicity, stability (thermodynamic and kinetic), pharmacokinetics and biodistribution. Proton relaxation enhancement (or relaxivity) is chiefly governed by the choice of metal and rotational correlation times.

The first feature to be considered during the design stage is the selection of the metal atom, which will dominate the measured relaxivity of the complex. Paramagnetic metal ions, as a result of their unpaired electrons, act as potent relaxation enhancement agents. They decrease the T₁ and T₂ relaxation times of nearby (r⁶ dependence) spins. Some paramagnetic ions decrease the T₁ without causing substantial linebroadening (e.g. gadolinium (III), (Gd³⁺)), while others induce drastic linebroadening (e.g. superparamagnetic iron oxide). The mechanism of T₁ relaxation is generally a through space dipole-dipole interaction between the unpaired electrons of the paramagnet (the metal atom with an unpaired electron) and bulk water molecules (water molecules that are not “bound” to the metal atom) that are in fast exchange with water molecules in the metal's inner coordination sphere (are bound to the metal atom).

For example, regions associated with a Gd³⁺ ion (near-by water molecules) appear bright in an MR image where the normal aqueous solution appears as dark background if the time between successive scans if the experiment is short (i.e. T₁ weighted image). Localized T₂ shortening caused by superparamagnetic particles is believed to be due to the local magnetic field inhomogeneities associated with the large magnetic moments of these particles. Regions associated with a superparamagnetic iron oxide particle appear dark in an MR image where the normal aqueous solution appears as high intensity background if the echo time (TE) in the spin-echo pulse sequence experiment is long (i.e. T₂ -weighted image). The lanthanide atom Gd³⁺ is by the far the most frequently chosen metal atom for MRI contrast agents because it has a very high magnetic moment (u²=63BM²), and a symmetric electronic ground state, (S⁸). Transition metals such as high spin Mn(II) and Fe(III) are also candidates due to their high magnetic moments.

Once the appropriate metal has been selected, a suitable ligand or chelate must be found to render the complex nontoxic. The term chelator is derived from the Greek word chele which means a “crabs claw”, an appropriate description for a material that uses its many “arms” to grab and hold on to a metal atom (see DTPA below). Several factors influence the stability of chelate complexes include enthalpy and entropy effects (e.g. number, charge and basicity of coordinating groups, ligand field and conformational effects). Various molecular design features of the ligand can be directly correlated with physiological results. For example, the presence of a single methyl group on a given ligand structure can have a pronounced effect on clearance rate. While the addition of a bromine group can force a given complex from a purely extracellular role to an effective agent that collects in hepatocytes.

Diethylenetriaminepentaacetic (DTPA) chelates and thus acts to detoxify lanthanide ions. The stability constant (K) for Gd(DTPA)²⁻ is very high (logK=22.4) and is more commonly known as the formation constant (the higher the logK, the more stable the complex). This thermodynamic parameter indicates the fraction of Gd³⁺ ions that are in the unbound state will be quite small and should not be confused with the rate (kinetic stability) at which the loss of metal occurs (k_{f}/k_{d}). The water soluble Gd(DTPA)²⁻ chelate is stable, nontoxic, and one of the most widely used contrast enhancement agents in experimental and clinical imaging research. It was approved for clinical use in adult patients in June of 1988. It is an extracellular agent that accumulates in tissue by perfusion dominated processes.

To date, a number of chelators have been used, including diethylenetriaminepentaacetic (DTPA), 1,4,7,10-tetraazacyclododecane′-N,N′N″,N′″-tetracetic acid (DOTA), and derivatives thereof. See U.S. Pat. Nos. 5,155,215, 5,087,440, 5,219,553, 5,188,816, 4,885,363, 5,358,704, 5,262,532, and Meyer et al., Invest. Radiol. 25: S53 (1990).

Image enhancement improvements using Gd(DTPA) are well documented in a number of applications (Runge et al., Magn, Reson. Imag. 3:85 (1991); Russell et al., AJR 152:813 (1989); Meyer et al., Invest. Radiol. 25:S53 (1990)) including visualizing blood-brain barrier disruptions caused by space occupying lesions and detection of abnormal vascularity. It has recently been applied to the functional mapping of the human visual cortex by defining regional cerebral hemodynamics (Belliveau et al., (1991) 254:719).

Another chelator used in Gd contrast agents is the macrocyclic ligand 1,4,7,10-tetraazacyclododecane-N,N′,N″N′″-tetracetic acid (DOTA). The Gd-DOTA complex has been thoroughly studied in laboratory tests involving animals and humans. The complex is conformationally rigid, has an extremely high formation constant (logK=28.5), and at physiological pH possess very slow dissociation kinetics. Recently, the GdDOTA complex was approved as an MRI contrast agent for use in adults and infants in France and has been administered to over 4500 patients.

Previous work has resulted in MRI contrast agents that report on physiologic or metabolic processes within a biological or other type of sample. As described in U.S. Pat. No. 5,707,605, PCT US96/08549, and U.S. Ser. No. 09/134,072, U.S. Pat. No. 5,980,862, U.S. Ser. No. 09/405,046, No. 09/866,512; No. 09/179,927, No. 09/716,175, PCT/US01/14665; U.S. Ser. No. 09/908,436; No. 09/972,302; U.S.S. No. 60/288,963; U.S.S. No. 60/282,136; U.S. Ser. No. 10/116,706 No. 60/285,602; and a utility application by Meade, entitled “Increasing In-Vivo Residence Time of AMR Contrast Agent”, filed Apr. 22, 2002; all of which are expressly incorporated herein by reference. MRI contrast agents have been constructed that allow an increase in contrast as a result of the interaction of a blocking moiety present on the agent with a target substance. That is, in the presence of the target substance, the exchange of water in one or more inner sphere coordination sites of the contrast agent is increased, leading to a brighter signal; in the absence of the target substance, the exchange of water is hindered and the image remains dark. Thus, the previous work enables imaging of physiological events rather than just structure.

The field of drug discovery and screening of drug candidates to identify lead compounds is rapidly expanding. Traditional approaches to identify and characterize new and useful drug candidates include the isolation of natural products or synthetic preparation, followed by testing against either known or unknown targets. See for example WO 94/24314, Gallop et al., J. Med. Chem. 37(9):1233 (1994); Gallop et al., J. Med. Chem. 37(10):1385 (1994); Ellman, Acc. Chem. Res. 29:132 (1996); Gordon et al., E. J. Med. Chem. 30:388s (1994); Gordon et al., Acc. Chem. Res. 29:144 (1996); WO 95/12608, all of which are incorporated by reference.

The screening of these libraries is done in a variety of ways, generally involving high throughput methods such as fluorescence activated cell screening (FACS), animal studies with the sacrifice of animals, etc.

However, there remains a need of a non-invasive way of doing research, drug studies and testing.

### SUMMARY OF THE INVENTION

In accordance with the above objects, the invention provides a library of MRI contrast agents comprising a chelate, a paramagnetic metal ion, and a different candidate agent. The candidate agent may be covalently attached to the chelate, or indirectly attached to the chelate via a linker. Suitable candidate agents for use in the present invention include peptides, carbohydrates, nucleic acids, and lipids.

In an additional aspect, the invention provides a library of MRI contrast agents further comprising a blocking moiety covalently attached to said chelator and/or a targeting moiety.

In an additional aspect, the invention provides methods of screening for binding interactions comprising providing a substrate with an immobilized target analyte, contacting the substrate with a library of contrast agents as described above, and detecting the presence of at least one contrast agent bound to the target analyte. Detection may be done by taking a magnetic resonance image of the substrate or by detecting fluorescence from the metal ion. In some embodiments, a plurality of target analytes are bound to the substrate.

In an additional aspect, the invention provides methods of screening for protease-activated MRI contrast agents comprising providing a substrate with a plurality of test sites, each test site comprising at least one MRI agent comprising a paramagnetic metal ion bound to a complex, the complex comprising a chelator and a peptide blocking moiety, adding a first protease to each of said test sites, taking an MRI image of said substrate wherein if an increase in image intensity is observed, said test site comprises a protease-activated contrast agent.

In an additional aspect, the invention provides of screening an animal comprising injecting the animal with a first MRI agent comprising a paramagnetic metal ion bound to a complex, said complex comprising a chelator and a first blocking moiety, taking a first MRI image, injecting the animal with a second MRI agent comprising a paramagnetic methal ion bound to a complex, said complex comprising a chelator and a second blocking moiety and taking a second MRI image. The animal may be pretreated with a drug candidate prior to imaging or be a transgenic animal.

In an additional aspect, the present invention provides a method of imaging gene expression of a garget gene in an animal comprising at a first time point, comprising injecting the animal with an MRI agent comprising a chelate, a paramagnetic metal ion and a moiety comprising a binding partner of the target protein of said target gene, taking a first MRI image of said animal, and then at a second time point, injecting said animal with said MRI agent, taking a second MRI image and comparing said first and second images to determine the course of gene expression.

In an additional aspect, the present invention provides a method of imaging disease progression in an animal comprising at a first time point, comprising injecting the animal with an MRI agent comprising a chelate, a paramagnetic metal ion and a moiety comprising a binding partner of the target protein correlated to said disease, taking a first MRI image of said animal, and then at a second time point, injecting said animal with said MRI agent, taking a second MRI image and comparing said first and second images to determine the course of gene expression. In some embodiments, the animal may be treated with a drug or be a transgenic animal.

### BRIEF DESCRIPTION OF THE DRAWINGS

FIG. 1 depicts a representative complex of the invention, where the blocking moiety is tethered at one end only. The blocking moiety comprises a enzyme substrate and a coordination site barrier. The R group is the coordination site barrier.

FIG. 2 depicts a representative complex of the invention, wherein the blocking moiety is tethered at two ends. The R group is the coordination site barrier.

FIGS. 3A, 3B, 3C, 3D, 3E, 3F, and 3G depict several of the possible conformations of the dimer embodiments. Boxes represent chelators, with M being the paramagnetic metal ions. FIGS. 3A and 3B represent two possible duplex conformations. In FIG. 3A, R₂₇ can be a linker, such as described herein as R₂₆, a cleavable moiety such as an enzyme substrate such as a peptide, or a blocking moiety that will preferentially interact with the target molecule. R₂₈, which may or may not be present depending on R₂₇, is a coordination site barrier similar to R₂₃ or a blocking moiety. FIG. 3B has R₂₈ blocking moieties or coordination site barriers attached via an R₂₇ group to two chelators. FIG. 3C is similar to FIG. 3A, but at least one of the R₂₇ groups must be a cleavable moiety. FIG. 3D depicts the case where two blocking moieties or coordination site barriers are present; if R₂₇ is a blocking moiety, R₂₈ need not be present. FIG. 3E is similar to 3B but the chelators need not be covalently attached. FIGS. 3F (single MRI agents) and 3G (duplex agents) are multimers of MRI contrast agents, wherein n can be from 1 to 1000, with from 1 to about 20 being preferred, and from about 1 to 10 being especially preferred. FIGS. 3H and 3I depict polymer 10 as defined herein being attached to either single MRI agents (3H) or duplex MRI agents (3I).

FIGS. 4A, 4B, 4C, 4D and 4E depict several different linkers that are cleavable by esterases. FIG. 4A depicts schematic esterase enzyme mechanisms. FIGS. 4B, 4C, 4D and 4E depict several different linkers, using a blocking moiety, although as will be appreciated, other blocking moieties can be used as well.

FIG. 5 depicts a synthetic scheme for the synthesis of the compositions of the invention that have peptides as a component of the system (either as a blocking moiety, linker or targeting moiety, for example).

FIG. 6 depicts a representative structure utilizing a blocking moiety comprising a peptide and a HIV-TAT peptide as a targeting moiety, attached with an aryl amine linker, although as outlined herein, any number of different linkers may be used.

FIG. 7 depicts a variety of configurations for adding targeting moieties to the compositions of the invention.

FIGS. 8A, 8B and 8C depict some embodiments of the invention. FIG. 8A depicts the situation wherein the drug 20 provides the coordination atom to the metal ion 10 (shown as Gd, herein) in the chelate 5, although the coordination atom may be contributed by a linker, a coordination site barrier, or a cleavage site (FIG. 8B). Upon exposure to the physiological target 30, a conformational change occurs, allowing the rapid exchange of water. FIG. 8B is similar, except a cleavage site 40 is used, which upon exposure to a cleavage agent cleaves off the drug 20. FIG. 8C depicts the use of a coordination site barrier 50, which in the absence of a cleavage agent such as an enzyme hinders the exchange of water. However, after cleavage, the target 30 is able to interact with the drug 20. Although not depicted, a targeting moiety may also be included in any of the embodiments herein; for example, a targeting moiety used to target the drug may attached to the drug 20 in FIG. 8A, either between the metal chelate and the drug or as a “terminal group” to the drug. Alternatively, the targeting moiety may be attached to the chelate 5 in another position. Similarly, a targeting moiety may be attached to the FIGS. 8B and 8C embodiments between the cleavage site 40 and the drug 20, or as a terminal group.

FIGS. 9A, 9B and 9C depict the use of coordination site barriers 60. A variety of conformations may be utilized as generally described in FIG. 8, including the use of targeting groups. It should also be noted that additional cleavage sites may be put into the system, for example to cleave the coordination site barrier 60 from the drug 20 in FIG. 9C.

FIGS. 10A-10H depict a number of different drugs that can be used as therapeutic blocking moieties. As will be appreciated by those in the art, and as described herein, any number of functional groups (either endogeneous to the structure or added exogeneously) can be used to attach these drugs, and their derivatives, to the chelates. FIG. 10A is docetaxel; 10B is etoposide; 10C is irinotecan; 10D is paclitaxel; 10E is tenoposide; 10F is is topotecan; 10G is vinblastine (note its derivative, vincristine); 10H is vindesine.

FIG. 11 depicts a preferred structure comprising a DOTA chelate, complexed with Gd+3, and comprising a doxorubicin therapeutic blocking moiety.

FIGS. 12A and 12B depict preferred structures comprising a DOTA chelate, complexed with Gd+3, and comprising a taxol therapeutic blocking moiety attached in two separate locations on the taxol. R1 in this case can be a variety of linkers, including esters, amides, cleavable linkers (particularly cleavable peptides such as DEVD), etc. In addition, while the therapeutic blocking moiety is shown attached to an “arm” of the DOTA chelate, attachment at the macrocycle is also done, as is outlined herein. Similarly, other moieties such as targeting moieties and other R substitution groups may be used.

FIG. 13 depicts a number of structures of the invention.

FIG. 14 depicts a number of X moieties of the invention.

FIG. 15 depicts several suitable coordination moieties.

FIGS. 16 A-D depict examples of imaging moieties of the present invention comprising a polymer and a plurality of MRI contrast agents attached to the polymer by a cleavable linker. CL=cleavable linker; BM=blocking moiety; TM=targeting moiety; M=paramagnetic ion; n is an integer of 1 or greater; and m is an integer of 0 or greater. CL is cleavable linker that either contributes a coordination atom, thereby serving as a blocking moiety (BM); or serves as a coordination site barrier.

FIG. 17A depicts a preferred chelate. In FIG. 17A each Q is independently selected from the group consisting of nitrogen, oxygen or sulfur; A-B is a structure selected from the group consisting of —CR₂—CR₂—, —CR═CR—, —CR₂—CR₂—CR₂—, —CR═CR—CR₂— and —CR₂—CR═CR—; X1 and X2 are independently selected from the group consisting of CR₂COO⁻, CR₂COOH, CR₂(BM), CR(CR₂COO⁻)₂, CR(CR₂COO⁻)(BM), CR(CR₂COOH)₂ and CR(CR₂COOH)(BM), wherein BM is a blocking moiety; and each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, targeting moiety, blocking moiety, or, together with an adjacent R group forms an alkyl or aryl group; wherein either: a) X¹ or X² comprises a BM; or b) at least one R comprises a BM.

FIG. 17B depicts a preferred chelate. In FIG. 17B, each Q is independently selected from the group consisting of nitrogen, oxygen or sulfur; A-B is a structure selected from the group consisting of —CR₂—CR₂—, —CR═CR—, —CR₂—CR₂—CR₂—, —CR═CR—CR₂— and —CR₂—CR═CR—; X³, X⁴, X⁵, X⁶ and X⁷ are independently selected from the group consisting of —(CR₂)nCOO⁻, —(CR₂₎ₙCOOH, (CR₂)n(BM), —CR(CR₂COO⁻)₂, −CR(CR₂COO⁻)(BM), —CR(CR₂COOH)₂ and —CR(CR₂COOH)(BM), —(CR₂)n—CR((CR₂)m—COOH)2, —(CR₂)n—CR((CR₂)m—COO⁻)2, —(CR₂)m—CR[((CR₂)m—COOH))((CR₂)m—BM), —(CR₂)n—C(CR₂)m—COOH)3; and —C((CR₂)n—COOH)3, wherein BM is a blocking moiety; each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group; wherein optionally two of X³-X⁷ are joined to form —CR₂—CR₂—, —CR═CR—, —CR₂—CR₂—CR₂— or —CR═CR—CR₂—; and wherein either: a) X³, X⁴, X⁵, X⁶ or X⁷ comprises a BM; or b) at least one R comprises a BM.

FIG. 17C depicts a preferred chelate. In FIG. 17C, each Q is independently selected from the group consisting of nitrogen, sulfur or oxygen; each Z is —(CR₂)n— wherein n is at least 1 and R is a substitution group; at least two of X⁸-X¹⁰ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n—CR((CR₂)m—COOH)2, —(CR₂)n—CR((CR₂)m—COO⁻)2, —(CR₂)m—CR[((CR₂)m—COOH))((CR₂)m—BM), —(CR₂)n—C(CR₂)m—COOH)3; and —C((CR₂)n—COOH)3; wherein at least one R or X comprises a BM.

FIG. 17D depicts a preferred chelate. In FIG. 17D, A and B are selected from the group consisting of CR₂—CR₂, CR═CR, CR₂—CR₂—CR₂, CR═CR—CR₂, and CR₂—CR═CR; each Q is independently selected from the group consisting of nitrogen, sulfur or oxygen; each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group; wherein X¹¹-X¹⁶ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n—CR((CR₂)m—COOH)2, —(CR₂)n—CR((CR₂)m—COO⁻)2, —(CR₂)m—CR[((CR₂)m—COOH))((CR₂)m—BM), —(CR₂)n—C(CR₂)m—COOH)3; and —C((CR₂)n—COOH)3; wherein optionally two of X¹-X⁷ are joined to form a C1-5 alkyl group; wherein either:

a) X¹¹, X¹², X¹³, X¹⁴, X¹⁵ or X¹⁶ comprises a BM; or b) at least one R comprises a BM.

FIG. 17E depicts a preferred chelate. In FIG. 17E, A and B are selected from the group consisting of CR₂—CR₂, CR═CR, CR₂—CR₂—CR₂, CR═CR—CR₂, and CR₂—CR═CR; each Q is independently selected from the group consisting of nitrogen, sulfur or oxygen; each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group; wherein each of X²³ are independently selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n—CR((CR₂)m—COOH)2, —(CR₂)n—CR((CR₂)m—COO⁻)2, —(CR₂)m—CR[((CR₂)m—COOH))((CR₂)m—BM), —-(CR₂)n—C(CR₂)m—COOH)3; and —C((CR₂)n—COOH)3; wherein optionally two of X¹⁷-X²³ are joined to form a C1-10 alkyl group; wherein either:

a) X¹⁷, X¹⁸, X¹⁹, X²⁰, X²¹, X²² or X²³ comprises a BM; or b) at least one R comprises a BM.

### DETAILED DESCRIPTION OF THE INVENTION

The present invention provides a variety of compositions and methods useful in the high throughput screening and analysis of drug candidates, disease progression, and gene expression monitoring. In general, the methods of the invention utilize two types of MRI contrast agents, referred to herein as “activatible” and “non-activatible”. That is, as outlined below, contrast agents can be constructed that are either always on (“non-activatible”) or that are “triggered” by the presence of physiological agents (“activatible”). These agents then find use in a variety of methods. For example, libraries of either activatible or non-activatible agents can be made for testing for binding to any number of different target analytes. Once an agent/target pair has been identified, magnetic resonance imaging and/or fluorescent studies can be run, that allow the elucidation of any variety of parameters, including the time courses of disease progression, gene expression, drug response and biodistribution of drugs. In addition, the methods of the invention find use in functional genomics applications, from gene discovery to the identification of validated targets to the development of lead drug candidates.

As outlined herein, there are two types of imaging agents that find use in the present invention. The first are standard MRI contrast agents (which, as outlined below, in some cases are used for fluorescent detection rather than MRI imaging), and activatible agents.

In a preferred embodiment, the present invention provides magnetic resonance imaging contrast agents which can detect physiological agents or target substances. The MRI agents of the invention are relatively inactive, or have weak relaxivity, as contrast enhancement agents in the absence of the physiological target substance, and are activated, thus altering the MR image, in the presence of the physiological target substance.

Viewed simplistically, this “trigger” mechanism, whereby the contrast agent is “turned on” (i.e. increases the relaxivity) by the presence of the target substance, is based on a dynamic equilibrium that affects the rate of exchange of water molecules in one or more coordination sites of a paramagnetic metal ion contained in the MRI contrast agents of the present invention. In turn, the rate of exchange of the water molecule is determined by the presence or absence of the target substance in the surrounding environment. Thus, in the absence of the target substance, the metal ion complexes of the invention which chelate the paramagnetic ion have reduced coordination sites available which can rapidly exchange with the water molecules of the local environment. In such a situation, the water coordination sites are substantially occupied or blocked by the coordination atoms of the chelator and at least one blocking moiety. Thus, the paramagnetic ion has essentially no water molecules in its “inner-coordination sphere”, i.e. actually bound to the metal when the target substance is absent. It is the interaction of the paramagnetic metal ion with the protons on the inner coordination sphere water molecules and the rapid exchange of such water molecules that cause the high observed relaxivity, and thus the imaging effect, of the paramagnetic metal ion. Accordingly, if all the coordination sites of the metal ion in the metal ion complex are occupied with moieties other than water molecules, as is the case when the target substance is absent, there is little if any net enhancement of the imaging signal by the metal ion complexes of the invention. However, when present, the target substance interacts with the blocking moiety or moities of the metal ion complex, effectively freeing at least one of the inner-sphere coordination sites on the metal ion complex. The water molecules of the local environment are then available to occupy the inner-sphere coordination site or sites, which will cause an increase in the rate of exchange of water and relaxivity of the metal ion complex toward water thereby producing image enhancement which is a measure of the presence of the target substance.

Generally, a 2 to 5% change in the MRI signal used to generate the image is sufficient to be detectable. Thus, it is preferred that the agents of the invention in the presence of a target substance increase the MRI signal by at least 2 to 5% as compared to the signal gain the absence of the target substance. Signal enhancement of 2 to 90% is preferred, and 10 to 50% is more preferred for each coordination site made available by the target substance interaction with the blocking moiety. That is, when the blocking moiety occupies two or more coordination sites, the release of the blocking moiety can result in double the increase in signal or more as compared to a single coordination site.

It should be understood that even in the absence of the target substance, at any particular coordination site, there will be a dynamic equilibrium for one or more coordination sites as between a coordination atom of the blocking moiety and water molecules. That is, even when a coordination atom is tightly bound to the metal, there will be some exchange of water molecules at the site. However, in most instances, this exchange of water molecules is neither rapid nor significant, and does not result in significant image enhancement. However, upon exposure to the target substance, the blocking moiety dislodges from the coordination site and the exchange of water is increased, i.e. rapid exchange and therefore an increase in relaxivity may occur, with significant image enhancement.

The complexes of the invention comprise a chelator and a blocking moiety. The metal ion complexes of the invention comprise a paramagnetic metal ion bound to a complex comprising a chelator and a blocking moiety. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd+3 or Gd(III)), iron III (Fe+3 or Fe(III)), manganese II (Mn+2 or Mn(II)), ytterbium III (Yb+3 or Yb(III)), dysprosium (Dy+3 or Dy(III)), and chromium (Cr(III) or Cr+3). In a preferred embodiment the paramagnetic ion is the lanthanide atom Gd(III), due to its high magnetic moment (u²=63BM2), a symmetric electronic ground state (S8), and its current approval for diagnostic use in humans.

In addition to the metal ion, the metal ion complexes of the invention comprise a chelator and a blocking moiety which may be covalently attached to the chelator. Due to the relatively high toxicity of many of the paramagnetic ions, the ions are rendered nontoxic in physiological systems by binding to a suitable chelator. Thus, the substitution of blocking moieties in coordination sites of the chelator, which in the presence of the target substance are capable of vacating the coordination sites in favor of water molecules, may render the metal ion complex more toxic by decreasing the half-life of dissociation for the metal ion complex. Thus, in a preferred embodiment, only a single coordination site is occupied or blocked by a blocking moiety. However, for some applications, e.g. analysis of tissue and the like, the toxicity of the metal ion complexes may not be of paramount importance. Similarly, some metal ion complexes are so stable that even the replacement of one or more additional coordination atoms with a blocking moiety does not significantly effect the half-life of dissociation. For example, DOTA, described below, when complexed with Gd(III) is extremely stable. Accordingly, when DOTA serves as the chelator, several of the coordination atoms of the chelator may be replaced with blocking moieties without a significant increase in toxicity. Additionally such an agent would potentially produce a larger signal since it has two or more coordination sites which are rapidly exchanging water with the bulk solvent.

There are a variety of factors which influence the choice and stability of the chelate metal ion complex, including enthalpy and entropy effects (e.g. number, charge and basicity of coordinating groups, ligand field and conformational effects).

In general, the chelator has a number of coordination sites containing coordination atoms which bind the metal ion. The number of coordination sites, and thus the structure of the chelator, depends on the metal ion. The chelators used in the metal ion complexes of the present invention preferably have at least one less coordination atom (n-1) than the metal ion is capable of binding (n), since at least one coordination site of the metal ion complex is occupied or blocked by a blocking moeity, as described below, to confer functionality on the metal ion complex. Thus, for example, Gd(III) may have 8 strongly associated coordination atoms or ligands and is capable of weakly binding a ninth ligand. Accordingly, suitable chelators for Gd(III) will have less than 9 coordination atoms. In a preferred embodiment, a Gd(III) chelator will have 8 coordination atoms, with a blocking moiety either occupying or blocking the remaining site in the metal ion complex. In an alternative embodiment, the chelators used in the metal ion complexes of the invention have two less coordination atoms (n-2) than the metal ion is capable of binding (n), with these coordination sites occupied by one or more blocking moieties. Thus, alternative embodiments utilize Gd(III) chelators with at least 5 coordination atoms, with at least 6 coordination atoms being preferred, at least 7 being particularly preferred, and at least 8 being especially preferred, with the blocking moiety either occupying or blocking the remaining sites. It should be appreciated that the exact structure of the chelator and blocking moiety may be difficult to determine, and thus the exact number of coordination atoms may be unclear. For example, it is possible that the chelator provide a fractional or non-integer number of coordination atoms; i.e. the chelator may provide 7.5 coordination atoms, i.e. the 8th coordination atom is on average not fully bound to the metal ion. However, the metal ion complex may still be functional, if the 8th coordination atom is sufficiently bound to prevent the rapid exchange of water at the site, and/or the blocking moiety impedes the rapid exchange of water at the site.

There are a large number of known macrocyclic chelators or ligands which are used to chelate lanthanide and paramagnetic ions. See for example, Alexander, Chem. Rev. 95:273-342 (1995) and Jackels, Pharm. Med. Imag, Section III, Chap. 20, p645 (1990), expressly incorporated herein by reference, which describes a large number of macrocyclic chelators and their synthesis. Similarly, there are a number of patents which describe suitable chelators for use in the invention, including U.S. Pat. Nos. 5,155,215, 5,087,440, 5,219,553, 5,188,816, 4,885,363, 5,358,704, 5,262,532, and Meyer et al., Invest. Radiol. 25: S53 (1990), all of which are also expressly incorportated by reference. Thus, as will be understood by those in the art, any of the known paramagnetic metal ion chelators or lanthanide chelators can be easily modified using the teachings herein to further comprise at least one blocking moiety.

When the metal ion is Gd(III), a preferred chelator is 1,4,7,10-tetraazacyclododecane-N,N′,N″, N′″-tetracetic acid (DOTA) or substituted DOTA. DOTA has the structure shown below:

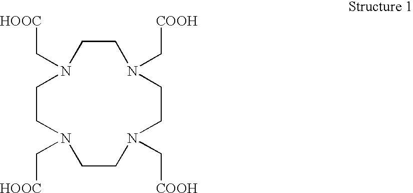

By “substituted DOTA” herein is meant that the DOTA may be substituted at any of the following positions, as shown below:

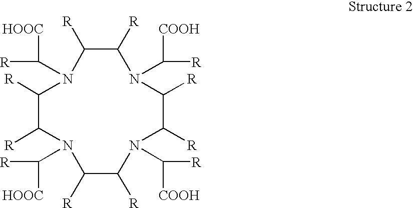

Suitable R substitution groups include a wide variety of groups, as will be understood by those in the art. For example, suitable substitution groups include substitution groups disclosed for DOTA and DOTA-type compounds in U.S. Pat. Nos. 5,262,532, 4,885,363, and 5,358,704. These groups include hydrogen, alkyl groups including substituted alkyl groups and heteroalkyl groups, aryl groups including substituted aryl and heteroaryl groups, phosphorus moieties, and blocking moieties. As will be appreciated by those skilled in the art, each position designated above may have two R groups attached (R′ and R″), although in a preferred embodiment only a single non-hydrogen R group is attached at any particular position; that is, preferably at least one of the R groups at each position is hydrogen. Thus, if R is an alkyl or aryl group, there is generally an additional hydrogen attached to the carbon, although not depicted herein. In a preferred embodiment, one R group is a blocking moiety and the other R groups are hydrogen.

By “alkyl group” or grammatical equivalents herein is meant a straight or branched chain alkyl group, with straight chain alkyl groups being preferred. If branched, it may be branched at one or more positions, and unless specified, at any position. Also included within the definition of alkyl are heteroalkyl groups, wherein the heteroatom is selected from nitrogen, oxygen, phosphorus, sulfur and silicon. Also included within the definition of an alkyl group are cycloalkyl groups such as C5 and C6 rings, and heterocycloalkyl.

Additional suitable heterocyclic substituted rings are depicted in U.S. Pat. No. 5,087,440, expressly incorporated by reference. In some embodiments, two adjacent R groups may be bonded together to form ring structures together with the carbon atoms of the chelator, such as is described in U.S. Pat. No. 5,358,704, expressly incorporated by reference. These ring structures may be similarly substituted.

The alkyl group may range from about 1 to 20 carbon atoms (C1-C20), with a preferred embodiment utilizing from about 1 to about 10 carbon atoms (C1-C10), with about C1 through about C5 being preferred. However, in some embodiments, the alkyl group may be larger, for example when the alkyl group is the coordination site barrier.

By “alkyl amine” or grammatical equivalents herein is meant an alkyl group as defined above, substituted with an amine group at any position. In addition, the alkyl amine may have other substitution groups, as outlined above for alkyl group. The amine may be primary (—NH₂R), secondary (—NHR₂), or tertiary (—NR₃). When the amine is a secondary or tertiary amine, suitable R groups are alkyl groups as defined above. A preferred alkyl amine is p-aminobenzyl. When the alkyl amine serves as the coordination site barrier, as described below, preferred embodiments utilize the nitrogen atom of the amine as a coordination atom, for example when the alkyl amine includes a pyridine or pyrrole ring.

By “aryl group” or grammatical equivalents herein is meant aromatic aryl rings such as phenyl, heterocyclic aromatic rings such as pyridine, furan, thiophene, pyrrole, indole and purine, and heterocyclic rings with nitrogen, oxygen, sulfur or phosphorus.

Included within the definition of “alkyl” and “aryl” are substituted alkyl and aryl groups. That is, the alkyl and aryl groups may be substituted, with one or more substitution groups. For example, a phenyl group may be a substituted phenyl group. Suitable substitution groups include, but are not limited to, halogens such as chlorine, bromine and fluorine, amines, hydroxy groups, carboxylic acids, nitro groups, carbonyl and other alkyl and aryl groups as defined herein. Thus, arylalkyl and hydroxyalkyl groups are also suitable for use in the invention. Preferred substitution groups include alkyl amines and alkyl hydroxy.

By “phosphorous moieties” herein is meant moieties containing the —PO(OH)(R₂₅)₂ group. The phosphorus may be an alkyl phosphorus; for example, DOTEP utilizes ethylphosphorus as a substitution group on DOTA. R₂₅ may be alkyl, substituted alkyl, hydroxy. A preferred embodiment has a —PO(OH)₂R₂₅ group.

The substitution group may also be hydrogen or a blocking moiety, as is described below.

In an alternative embodiment, when the metal ion is Gd(III), a preferred chelator is diethylenetriaminepentaacetic acid (DTPA) or substituted DTPA. DPTA has the structure shown below:

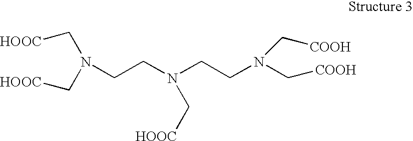

By “substituted DPTA” herein is meant that the DPTA may be substituted at any of the following positions, as shown below:

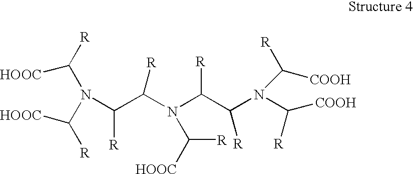

See for example U.S. Pat. No. 5,087,440.

Suitable R substitution groups include those outlined above for DOTA. Again, those skilled in the art will appreciate that there may be two R groups (R′ and R″) at each position designated above, although as described herein, at least one of the groups at each position is hydrogen, which is generally not depicted herein.

In an alternative embodiment, when the metal ion is Gd(III), a preferred chelator is 1,4,7,10-tetraazacyclododecane-N,N′,N″,N′″-tetraethylphosphorus (DOTEP) or substituted DOTEP (see U.S. Pat. No. 5,188,816). DOTEP has the structure shown below:

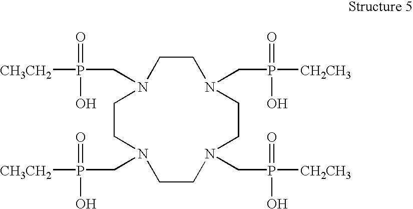

DOTEP may have similar R substitution groups as outlined above.

Other suitable Gd(III) chelators are described in Alexander, supra, Jackels, supra, U.S. Pat. Nos. 5,155,215, 5,087,440, 5,219,553, 5,188,816, 4,885,363, 5,358,704, 5,262,532, and Meyer et al., Invest. Radiol. 25: S53 (1990), among others.

When the paramagnetic ion is Fe(III), appropriate chelators will have less than 6 coordination atoms, since Fe(III) is capable of binding 6 coordination atoms. Suitable chelators for Fe(III) ions are well known in the art, see for example Lauffer et al., J. Am. Chem. Soc. 109:1622 (1987); Lauffer, Chem. Rev. 87:901-927 (1987); and U.S. Pat. Nos. 4,885,363, 5,358,704, and 5,262,532, all which describe chelators suitable for Fe(III).

When the paramagnetic ion is Mn(II) (Mn+2), appropriate chelators will have less than 5 or 6 coordination atoms, since Mn(II) is capable of binding 6 or 7 coordination atoms. Suitable chelators for Mn(II) ions are well known in the art; see for example Lauffer, Chem. Rev. 87:901-927 (1987) and U.S. Pat. Nos. 4,885,363, 5,358,704, and 5,262,532.

When the paramagnetic ion is Yb(III), appropriate chelators will have less than 7 or 8 coordination atoms, since Yt(III) is capable of binding 8 or 9 coordination atoms. Suitable chelators for Yt(III) ions include, but are not limited to, DOTA and DPTA and derivatives thereof (see Moi et al., J. Am. Chem. Soc. 110:6266-6267 (1988)) and those chelators described in U.S. Pat. No. 4,885,363 and others, as outlined above.

When the paramagnetic ion is Dy+3 (Dy(III)), appropriate chelators will have less than 7 or 8 coordination atoms, since DyIII is capable of binding 8 or 9 coordination atoms. Suitable chelators are known in the art, as above.

In a preferred embodiment, the chelator and the blocking moiety are covalently linked; that is, the blocking moiety is a substitution group on the chelator. In this embodiment, the substituted chelator, with the bound metal ion, comprises the metal ion complex which in the absence of the target substance has all possible coordination sites occupied or blocked; i.e. it is coordinatively saturated.

In an alternative embodiment, the chelator and the blocking moiety are not covalently attached. In this embodiment, the blocking moiety has sufficient affinity for the metal ion to prevent the rapid exchange of water molecules in the absence of the target substance. However, in this embodiment the blocking moiety has a higher affinity for the target substance than for the metal ion. Accordingly, in the presence of the target substance, the blocking moiety will have a tendency to be dislodged from the metal ion to interact with the target substance, thus freeing up a coordination site in the metal ion complex and allowing the rapid exchange of water and an increase in relaxivity.

What is important is that the metal ion complex, comprising the metal ion, the chelator and the blocking moiety, is not readily able to rapidly exchange water molecules when the blocking moeities are in the inner coordination sphere of the metal ion, such that in the absence of the target substance, there is less or little substantial image enhancement.

By “blocking moiety” or grammatical equivalents herein is meant a functional group associated with the chelator metal ion complexes of the invention which is capable of interacting with a target substance and which is capable, under certain circumstances, of substantially blocking the exchange of water in at least one inner coordination site of the metal ion of the metal ion complex. For example, when bound to or associated with the metal ion complexes of the invention, the blocking moiety occupies or blocks at least one coordination site of the metal ion in the absence of the target substance. Thus, the metal ion is coordinately saturated with the chelator and the blocking moiety or moieties in the absence of the target substance.

A blocking moiety may comprise several components. The blocking moiety has a functional moiety which is capable of interacting with a target substance, as outlined below. This functional moiety may or may not provide the coordination atom(s) of the blocking moiety. In addition, blocking moieties may comprise one or more linker groups to allow for correct spacing and attachment of the components of the blocking moiety. Furthermore, in the embodiment where the functional group of the blocking moiety does not contribute a coordination atom, the blocking moiety may comprise a coordination site barrier, which serves to either provide a coordination site atom or sterically prevent the rapid exchange of water at the coordination site; i.e. the coordination site barrier may either occupy or block the coordination site.

By “capable of interacting with a target substance” herein is meant that the blocking moiety has an affinity for the target substance, such that the blocking moiety will stop blocking or occupying at least one coordination site of the metal ion complex when the target substance is present. Thus, as outlined above, the blocking moiety is blocking or occupying at least one coordination site of the metal ion in the absence of the target substance. However, in the presence of the target substance, the blocking moiety associates or interacts with the target substance and is released from its association with the metal ion, thus freeing at least one coordination site of the metal ion such that the rapid exchange of water can occur at this site, resulting in image enhancement.

The nature of the interaction between the blocking moiety and the target substance will depend on the target substance to be detected or visualized via MRI. For example, suitable target substances include, but are not limited to, enzymes; proteins; peptides; nucleic acids; ions such as Ca+2, Mg+2, Zn+2, K+, Cl−, and Na+; cAMP; receptors such as cell-surface receptors and ligands; hormones; antigens; antibodies; ATP; NADH; NADPH; FADH₂; FNNH₂; coenzyme A (acyl CoA and acetyl CoA); and biotin, among others.

As will be appreciated by those skilled in the art, the possible enzyme target substances are quite broad. The target substance enzyme may be chosen on the basis of a correlation to a disease condition, for example, for diagnostic purposes. Alternatively, the metal ion complexes of the present invention may be used to establish such correlations.

Suitable classes of enzymes include, but are not limited to, hydrolases such as proteases, carbohydrases, lipases and nucleases; isomerases such as racemases, epimerases, tautomerases, or mutases; transferases, kinases and phophatases.

As will be appreciated by those skilled in the art, the potential list of suitable enzyme targets is quite large. Enzymes associated with the generation or maintenance of arterioschlerotic plaques and lesions within the circulatory system, inflammation, wounds, immune response, tumors, may all be detected using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; metalloproteinases including MMP-1 through MMP-10, particularly MMP-1, MMP-2, MMP-7 and MMP-9; and caspases, such as caspase-3, -5, -8 and other caspases of the apoptotic pathway, and interleukin-converting enzyme (ICE). Similarly, bacterial and viral infections may be detected via characteristic bacterial and viral enzymes. As will be appreciated in the art, this list is not meant to be limiting.

Once the target enzyme is identified or chosen, enzyme substrate blocking moieties can be designed using well known parameters of enzyme substrate specificities.

For example, when the enzyme target substance is a protease, the blocking moiety may be a peptide or polypeptide which is capable of being cleaved by the target protease, as defined below.

Preferred target substance/peptide blocking moiety pairs include, but are not limited to, cat B and GGGF; cat B and GFQGVQFAGF; cat B and GFGSVGFAGF; cat B and GLVGGAGAGF; cat B and GGFLGLGAGF; cat D and GFGSTFFAGF; caspase-3 and DEVD; MMP-7 and PELR; MMP-7 and PLGLAR; MMP-7 and PGLWA-(D-arg); MMP-7 and PMALWMR; and MMP-7 and PMGLRA.

Similarly, when the enzyme target substance is a carbohydrase, the blocking moiety will be a carbohydrate group which is capable of being cleaved by the target carbohydrase. For example, when the enzyme target is lactase or β-galactosidase, the enzyme substrate blocking moiety is lactose or galactose. Similar enzyme/blocking moiety pairs include sucraselsucrose, maltase/maltose, and α-amylase/amylose. In addition, the addition of carbohydrate moieties such as galactose, outlined herein, can alter the biodistribution of the agents; for example, the galactose blocking moieties outlined herein cause concentration in liver, kidneys and spleen.

Physiological target substances include enzymes and proteins associated with a wide variety of viruses including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like.

Similarly, bacterial targets can come from a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhil; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like.

In a preferred embodiment, the physiological target protein is an enzyme. As will be appreciated by those skilled in the art, the possible enzyme target substances are quite broad. Suitable classes of enzymes include, but are not limited to, hydrolases such as proteases, carbohydrases, lipases and nucleases; isomerases such as racemases, epimerases, tautomerases, or mutases; transferases, kinases and phophatases. Enzymes associated with the generation or maintenance of arterioschlerotic plaques and lesions within the circulatory system, inflammation, wounds, immune response, tumors, apoptosis, exocytosis, etc. may all be treated using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, such as interleukin-converting enzyme (ICE). Similarly, bacterial and viral infections may be detected via characteristic bacterial and viral enzymes. As will be appreciated in the art, this list is not meant to be limiting.

In some embodiments, the nature of the interaction is irreversible, such that the blocking moiety does not reassociate to block or occupy the coordination site; for example, when the blocking moiety comprises an enzyme substrate which is cleaved upon exposure to the target enzyme. Alternatively, the nature of the interaction is reversible, such that the blocking moiety will reassociate with the complex to hinder the exchange of water; for example, when the blocking moiety comprises an ion ligand, or a receptor ligand, as outlined below.

The corresponding blocking moieties will be enzyme substrates or inhibitors, receptor ligands, antibodies, antigens, ion binding compounds, substantially complementary nucleic acids, nucleic acid binding proteins, etc.

In a preferred embodiment, the target substance is an enzyme, and the blocking moiety is an enzyme substrate. In this embodiment, the blocking moiety is cleaved from the metal ion complex of the invention, allowing the exchange of water in at least one coordination site of the metal ion complex. This embodiment allows the amplification of the image enhancement since a single molecule of the target substance is able to generate many activated metal ion complexes, i.e. metal ion complexes in which the blocking moiety is no longer occupying or blocking a coordination site of the metal ion.

As will be appreciated by those skilled in the art, the possible enzyme target substances are quite broad. The target substance enzyme may be chosen on the basis of a correlation to a disease condition, for example, for diagnositic purposes. Alternatively, the metal ion complexes of the present invention may be used to establish such correlations.

Suitable classes of enzymes include, but are not limited to, hydrolases such as proteases, carbohydrases, lipases and nucleases; isomerases such as racemases, epimerases, tautomerases, or mutases; transferases, kinases and phophatases.

As will be appreciated by those skilled in the art, the potential list of suitable enzyme targets is quite large. Enzymes associated with the generation or maintenance of arterioschlerotic plaques and lesions within the circulatory system, inflammation, wounds, immune response, tumors, may all be detected using the present invention. Enzymes such as lactase, maltase, sucrase or invertase, cellulase, a-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain;; metalloproteinases including MMP-1 through MMP-10, particularly MMP-1, MMP-2, MMP-7 and MMP-9; and caspases, such as caspase-3, -5, -8 and other caspases of the apoptotic pathway, and interleukin-converting enzyme (ICE). Similarly, bacterial and viral infections may be detected via characteristic bacterial and viral enzymes. As will be appreciated in the art, this list is not meant to be limiting.

Once the target enzyme is identified or chosen, enzyme substrate blocking moieties can be designed using well known parameters of enzyme substrate specificities.

For example, when the enzyme target substance is a protease, the blocking moieity may be a peptide or polypeptide which is capable of being cleaved by the target protease. By “peptide” or “polypeptide” herein is meant a compound of about 2 to about 15 amino acid residues covalently linked by peptide bonds. Preferred embodiments utilize polypeptides from about 2 to about 8 amino acids, with about 2 to about 4 being the most preferred. Preferably, the amino acids are naturally occurring amino acids, although amino acid analogs and peptidomimitic structures are also useful. Under certain circumstances, the peptide may be only a single amino acid residue.

Preferred target substance/peptide blocking moiety pairs include, but are not limited to, cat B and GGGF; cat B and GFQGVQFAGF; cat B and GFGSVGFAGF; cat B and GLVGGAGAGF; cat B and GGFLGLGAGF; cat D and GFGSTFFAGF; caspase-3 and DEVD; MMP-7 and PELR; MMP-7 and PLGLAR; MMP-7 and PGLWA-(D-arg); MMP-7 and PMALWMR; and MMP-7 and PMGLRA.

Similarly, when the enzyme target substance is a carbohydrase, the blocking moiety will be a carbohydrate group which is capable of being cleaved by the target carbohydrase. For example, when the enzyme target is lactase or β-galactosidase, the enzyme substrate blocking moiety is lactose or galactose. Similar enzyme/blocking moiety pairs include sucrase/sucrose, maltase/maltose, and α-amylase/amylose. In addition, the addition of carbohydrate moieties such as galactose, outlined herein, can alter the biodistribution of the agents; for example, the galactose blocking moieties outlined herein cause concentration in liver, kidneys and spleen.

In another embodiment, the blocking moiety may be an enzyme inhibitor, such that in the presence of the enzyme, the inhibitor blocking moiety disassociates from the metal ion complex to interact or bind to the enzyme, thus freeing an inner coordination sphere site of the metal ion for interaction with water. As above, the enzyme inhibitors are chosen on the basis of the enzyme target substance and the corresponding known characteristics of the enzyme.

In a preferred embodiment, the blocking moiety is a phosphorus moiety, as defined above, such as —(OPO(OR₂))ₙ, wherein n is an integer from 1 to about 10, with from 1 to 5 being preferred and 1 to 3 being particularly preferred. Each R is independently hydrogen or a substitution group as defined herein, with hydrogen being preferred. This embodiment is particularly useful when the target molecule is alkaline phosphatase or a phosphodiesterase, or other enzymes known to cleave phosphorus containing moieties such as these.

In one embodiment, the blocking moiety is a nucleic acid. The nucleic acid may be single-stranded or double stranded, and includes nucleic acid analogs such as peptide nucleic acids and other well-known modifications of the ribose-phosphate backbone, such as phosphorthioates, phosphoramidates, morpholino structures, etc. The target molecule can be a substantially complementary nucleic acid or a nulceic acid binding moiety, such as a protein.

In a preferred embodiment, the target substance is a physiological agent. As for the enzyme/substrate embodiment, the physiological agent interacts with the blocking moiety of the metal ion complex, such that in the presence of the physiological agent, there is rapid exchange of water in at least one inner sphere coordination site of the metal ion complex. Thus, the target substance may be a physiologically active ion, and the blocking moiety is an ion binding ligand. For example, as shown in the Examples, the target substance may be the Ca+2 ion, and the blocking moiety may be a calcium binding ligand such as is known in the art (see Grynkiewicz et al., J. Biol. Chem. 260(6):3440-3450 (1985); Haugland, R. P., Molecular Probes Handbook of Fluorescent Probes and Research Chemicals (1989-1991)). Other suitable target ions include Mn+2, Mg+2, Zn+2, Na+, and Cl−.

When Ca+2 is the target substance, preferred blocking moieties include, but are not limited to, the acetic acid groups of bis(o-amino-phenoxy)ethane-N,N,N′,N′-tetraacetic acid (BAPTA), ethylene glycol bis(β-aminoethyl ether)-N,N,N′,N′-tetraacetic acid (EGTA); ethylenediaminetetracetic acid (EDTA); and derivatives thereof, such as disclosed in Tsien, Biochem. 19:2396-2404 (1980). Other known chelators of Ca+2 and other divalent ions, such as quin2 (2-[[2-[bis(carboxymethyl)amino]-5-methylphenoxy]methyl-6-methoxy-8-[bis(carboxymethyl)amino]quinoline; fura-1, fura-2, fura-3, stil-1, stil-2 and indo-1 (see Grynkiewicz et al., supra).

As for the enzyme/substrate embodiments, the metabolite may be associated with a particular disease or condition within an animal. For example, as outlined below, BAPTA-DOTA derivatives may be used to diagnose Alzeheimer's disease and other neurological disorders.

In a preferred embodiment, the blocking moiety is a ligand for a cell-surface receptor or is a ligand which has affinity for a extracellular component. In this embodiment, as for the physiological agent embodiment, the ligand has sufficient affinity for the metal ion to prevent the rapid exchange of water molecules in the absence of the target substance. Alternatively, there may be R groups “locking” the ligand into place, as described herein, resulting in either the contribution of a coordination atom or that the ligand serves as a coordination site barrier. In this embodiment the ligand blocking moiety has a higher affinity for the target substance than for the metal ion. Accordingly, in the presence of the target substance, the ligand blocking moiety will interact with the target substance, thus freeing up at least one coordination site in the metal ion complex and allowing the rapid exchange of water and an increase in relaxivity. Additionally, in this embodiment, this may result in the accumulation of the MRI agent at the location of the target, for example at the cell surface. This may be similar to the situation where the blocking moiety is an enzyme inhibitor, as well.

In a preferred embodiment, the blocking moiety is a photocleavable moiety. That is, upon exposure to a certain wavelength of light, the blocking moiety is cleaved, allowing an increase in the exchange rate of water in at least one coordination site of the complex. This embodiment has particular use in developmental biology fields (cell lineage, neuronal development, etc.), where the ability to follow the fates of particular cells is desirable. Suitable photocleavable moieties are similar to “caged” reagents which are cleaved upon exposure to light. A particularly preferred class of photocleavable moieties are the O-nitrobenzylic compounds, which can be synthetically incorporated into a blocking moiety via an ether, thioether, ester (including phosphate esters), amine or similar linkage to a heteroatom (particularly oxygen, nitrogen or sulfur). Also of use are benzoin-based photocleavable moieties. A wide variety of suitable photocleavable moieties is outlined in the Molecular Probes Catalog, supra.

In a preferred embodiment, the compounds have a structure depicted below in Structure 18, which depicts a nitrobenzyl photocleavable group, although as will be appreciated by those in the art, a wide variety of other moieties may be used:

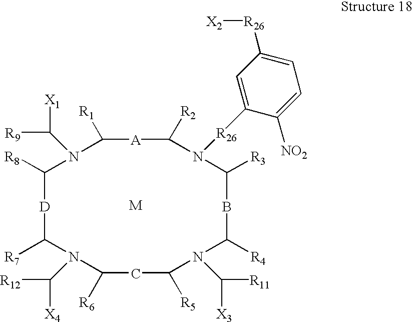

Structure 18 depicts a DOTA-type chelator, although as will be appreciated by those in the art, other chelators may be used as well. R₂₆ is a linker as defined below. Similarly, the X₂ group may be as defined above, although additional structures may be used, for example a coordination site barrier as outlined herein. Similarly, there may be substitutent groups on the aromatic ring, as is known in the art.

The blocking moiety itself may block or occupy at least one coordination site of the metal ion. That is, one or more atoms of the blocking moiety (i.e. the enzyme substrate, ligand, moiety which interacts with a physiological agent, photocleavable moiety, etc.) itself serves as a coordination atom, or otherwise blocks access to the metal ion by steric hinderance. For example, it appears that one or more of the atoms of the galactose blocking moiety outlined in the Examples may be direct coordination atoms for the Gd(III) metal ion. Similarly, peptide based blocking moieties for protease targets may contribute coordination atoms.

In an alternative embodiment, the blocking moiety further comprises a “coordination site barrier” which is covalently tethered to the complex in such a manner as to allow disassociation upon interaction with a target substance. For example, it may be tethered by one or more enzyme substrate blocking moieties. In this embodiment, the coordination site barrier blocks or occupies at least one of the coordination sites of the metal ion in the absence of the target enzyme substance. Coordination site barriers are used when coordination atoms are not provided by the functional portion of the blocking moiety, i.e. the component of the blocking moiety which interacts with the target substance. The blocking moiety or moieties such as an enzyme substrate serves as the tether, covalently linking the coordination site barrier to the metal ion complex. In the presence of the enzyme target, the enzyme cleaves one or more of the enzyme substrates, either within the substrate or at the point of attachment to the metal ion complex, thus freeing the coordination site barrier. The coordination site or sites are no longer blocked and the bulk water is free to rapidly exchange at the coordination site of the metal ion, thus enhancing the image. As will be appreciated by those in the art, a similar result can be accomplished with other types of blocking moieties.

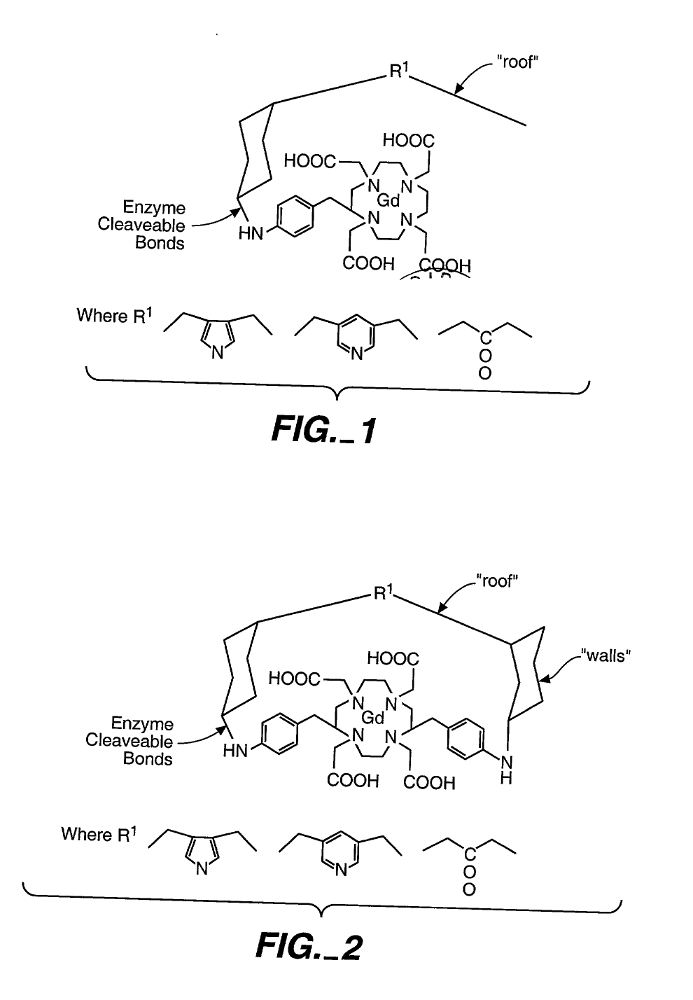

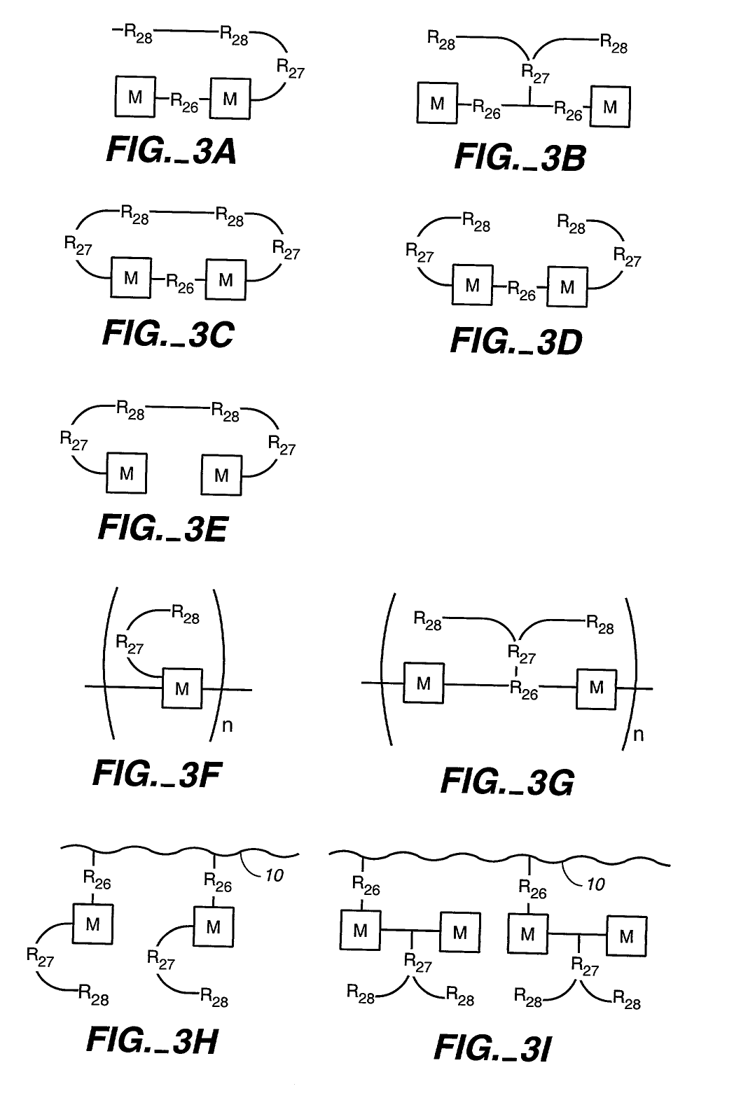

In one embodiment, the coordination site barrier is attached to the metal ion complex at one end, as is depicted in FIG. 1. When the enzyme target cleaves the substrate blocking moiety, the coordination site barrier is released. In another embodiment, the coordination site barrier is attached to the metal ion complex with more than one substrate blocking moiety, as is depicted in FIG. 2 for two attachments. The enzyme target may cleave only one side, thus removing the coordination site barrier and allowing the exchange of water at the coordination site, but leaving the coordination site barrier attached to the metal ion complex. Alternatively, the enzyme may cleave the coordination site barrier completely from the metal ion complex.

In a preferred embodiment, the coordination site barrier occupies at least one of the coordination sites of the metal ion. That is, the coordination site barrier contains at least one atom which serves as at least one coordination atom for the metal ion. In this embodiment, the coordination site barrier may be a heteroalkyl group, such as an alkyl amine group, as defined above, including alkyl pyridine, alkyl pyrroline, alkyl pyrrolidine, and alkyl pyrole, or a carboxylic or carbonyl group. The portion of the coordination site barrier which does not contribute the coordination atom may also be consider a linker group. Preferred coordination site barriers are depicted in FIG. 2.

In an alternative embodiment, the coordination site barrier does not directly occupy a coordination site, but instead blocks the site sterically. In this embodiment, the coordination site barrier may be an alkyl or substituted group, as defined above, or other groups such as peptides, proteins, nucleic acids, etc.

In this embodiment, the coordination site barrier is preferrably linked via two enzyme substrates to opposite sides of the metal ion complex, effectively “stretching” the coordination site barrier over the coordination site or sites of the metal ion complex, as is depicted in FIG. 2.

In some embodiments, the coordination site barrier may be “stretched” via an enzyme substrate on one side, covalently attached to the metal ion complex, and a linker moeity, as defined below, on the other. In an alternative embodiment, the coordination site barrier is linked via a single enzyme substrate on one side; that is, the affinity of the coordination site barrier for the metal ion is higher than that of water, and thus the blocking moiety, comprising the coordination site barrier and the enzyme substrate, will block or occupy the available coordination sites in the absence of the target enzyme.

In some embodiments, the metal ion complexes of the invention have a single associated or bound blocking moiety. In such embodiments, the single blocking moiety impedes the exchange of water molecules in at least one coordination site. Alternatively, as is outlined below, a single blocking moiety may hinder the exchange of water molecules in more than one coordination site, or coordination sites on different chelators.

In alternative embodiments, two or more blocking moieties are associated with a single metal ion complex, to implede the exchange of water in at least one or more coordination sites.

It should be appreciated that the blocking moieties of the present invention may further comprise a linker group as well as a functional blocking moiety. That is, blocking moieties may comprise functional blocking moieties in combination with a linker group and/or a coordination site barrier.

Linker groups (sometimes depicted herein as R₂₆) will be used to optimize the steric considerations of the metal ion complex. That is, in order to optimize the interaction of the blocking moiety with the metal ion, linkers may be introduced to allow the functional blocking moiety to block or occupy the coordination site. In general, the linker group is chosen to allow a degree of structural flexibility. For example, when a blocking moiety interacts with a physiological agent which does not result in the blocking moiety being cleaved from the complex, the linker must allow some movement of the blocking moiety away from the complex, such that the exchange of water at at least one coordination site is increased.

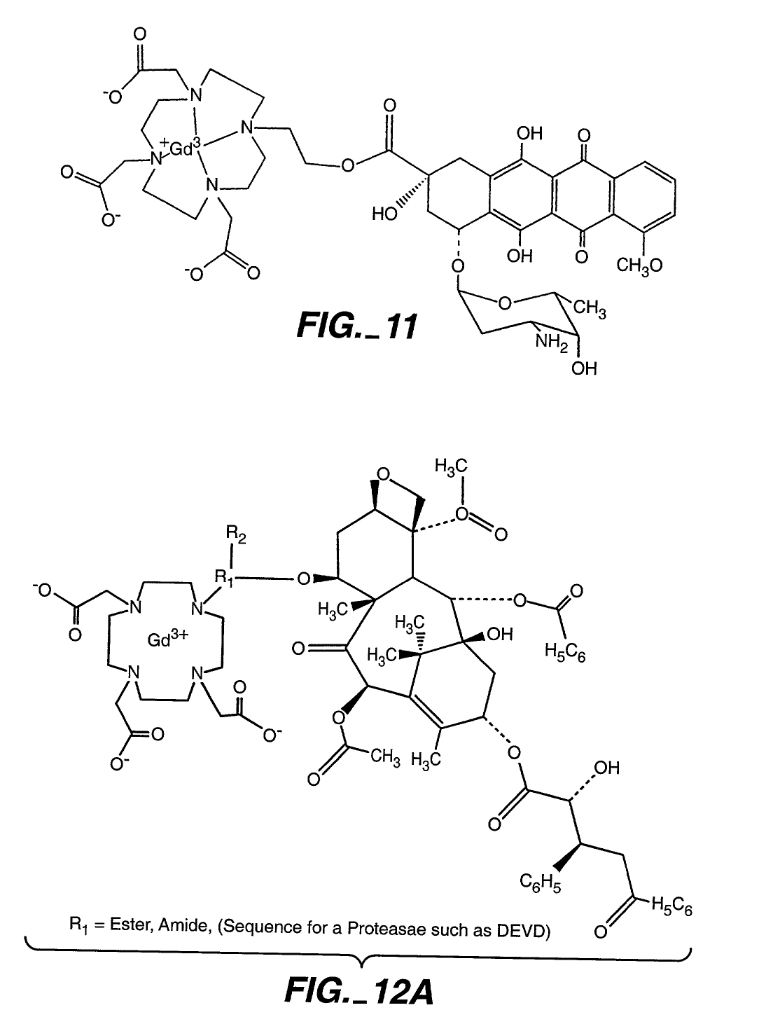

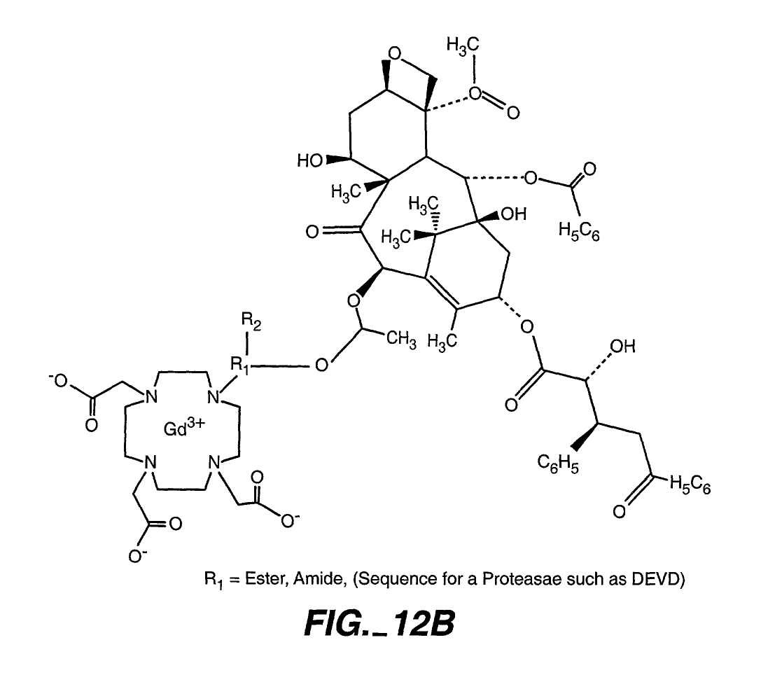

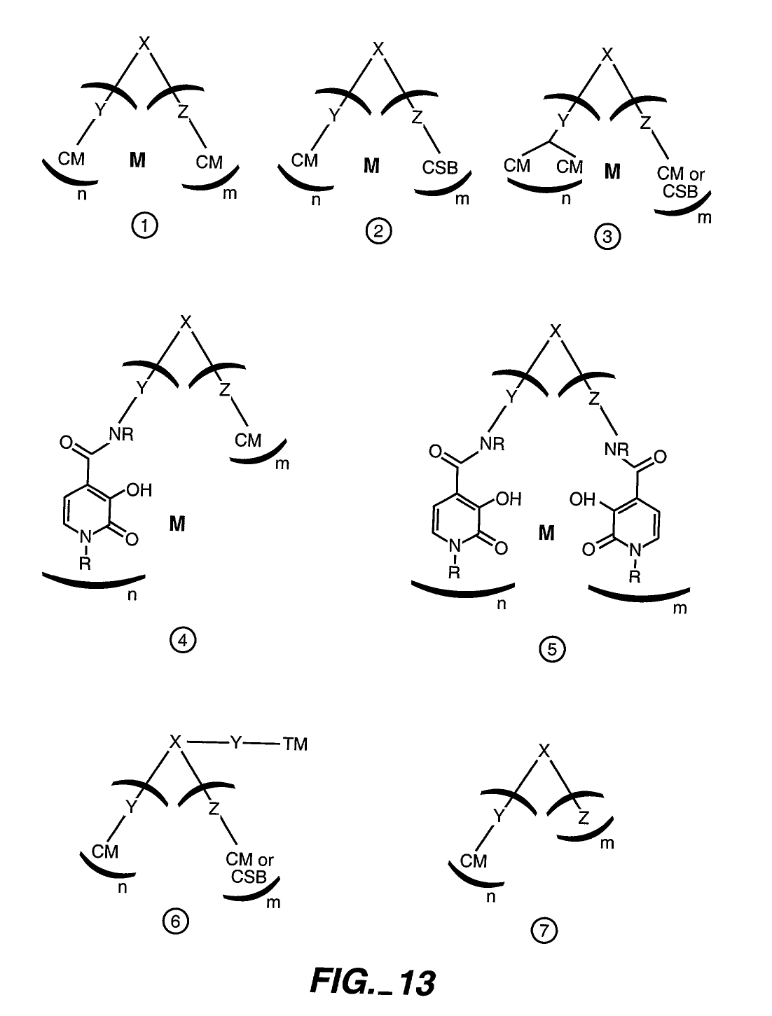

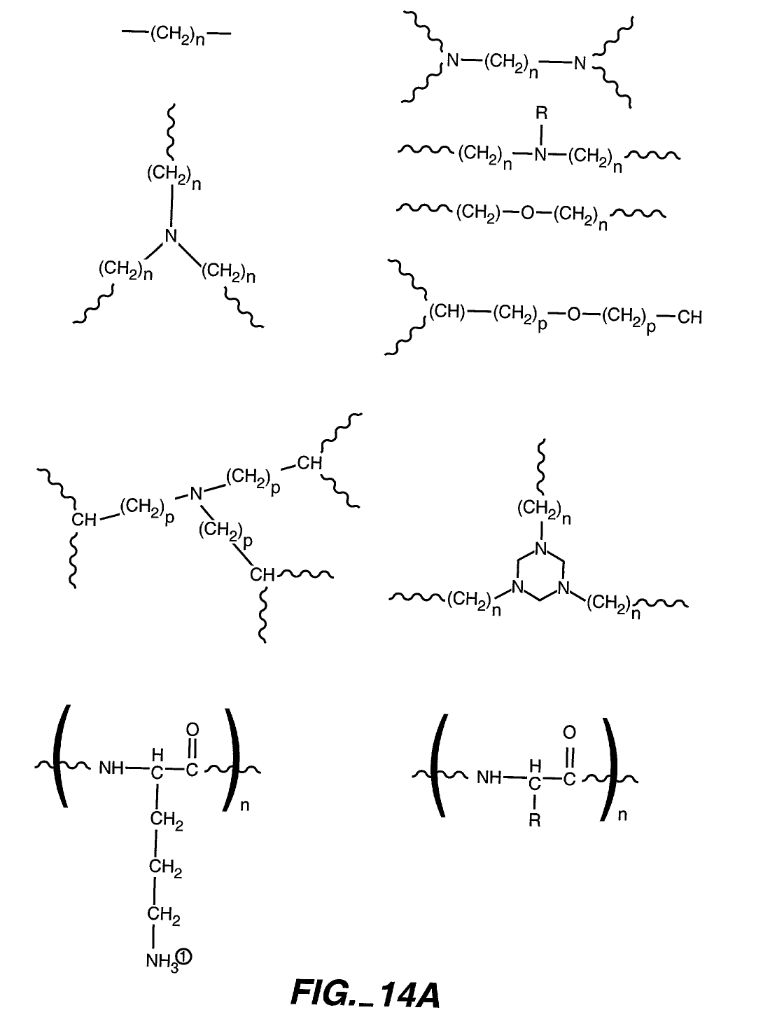

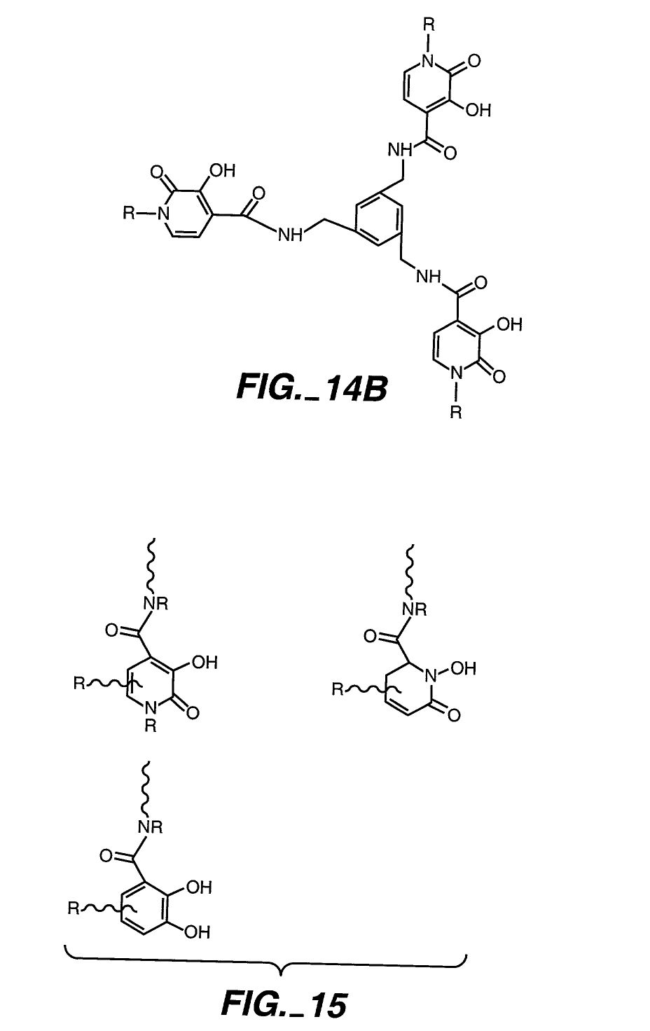

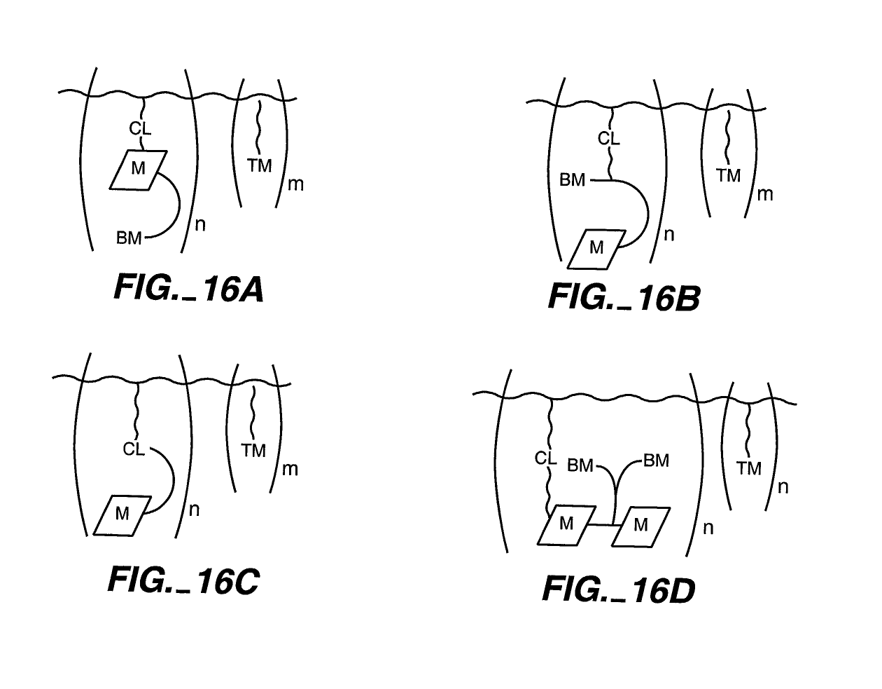

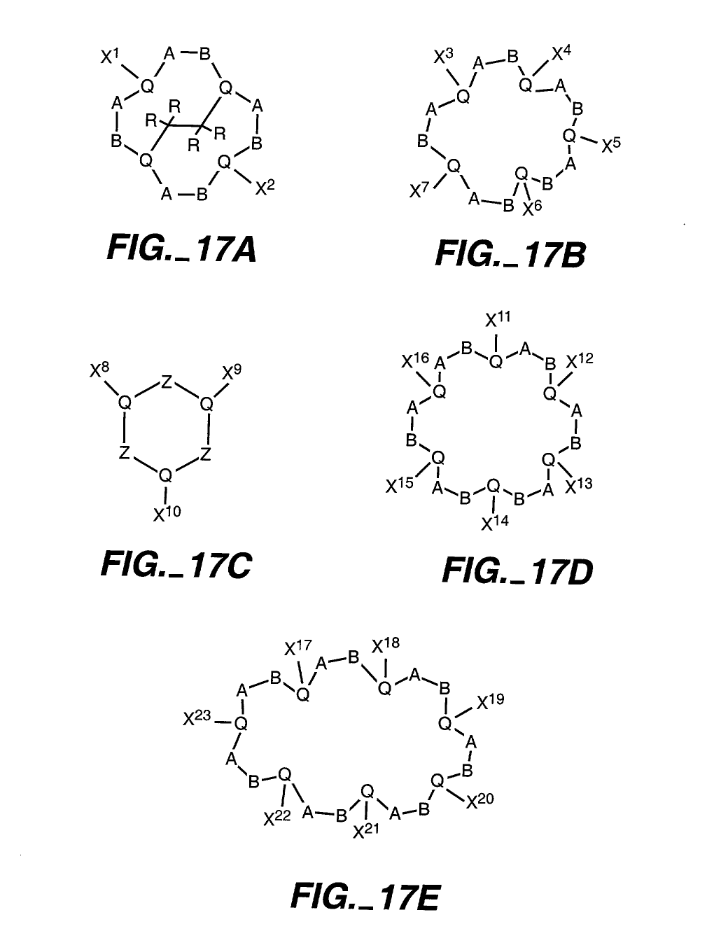

Generally, suitable linker groups include, but are not limited to, alkyl and aryl groups, including substituted alkyl and aryl groups and heteroalkyl (particularly oxo groups) and heteroaryl groups, including alkyl amine groups, as defined above. Preferred linker groups include p-aminobenzyl, substituted p-aminobenzyl, diphenyl and substituted diphenyl, alkyl furan such as benzylfuran, carboxy, and straight chain alkyl groups of 1 to 10 carbons in length. Particularly preferred linkers include p-aminobenzyl, methyl, ethyl, propyl, butyl, pentyl, hexyl, acetic acid, propionic acid, aminobutyl, p-alkyl phenols, 4-alkylimidazole. The selection of the linker group is generally done using well known molecular modeling techniques, to optimize the obstruction of the coordination site or sites of the metal ion. In addition, as outlined in the Examples, the length of this linker may be very important in order to achieve optimal results. As shown in FIG. 11, the length of the linker, i.e the spacer between the chelator and the coordination atom(s) of the blocking moiety, contributes to the steric conformation and association of the coordination atoms with the metal ion, thus allowing excellent blocking of the metal ion by the blocking moiety.

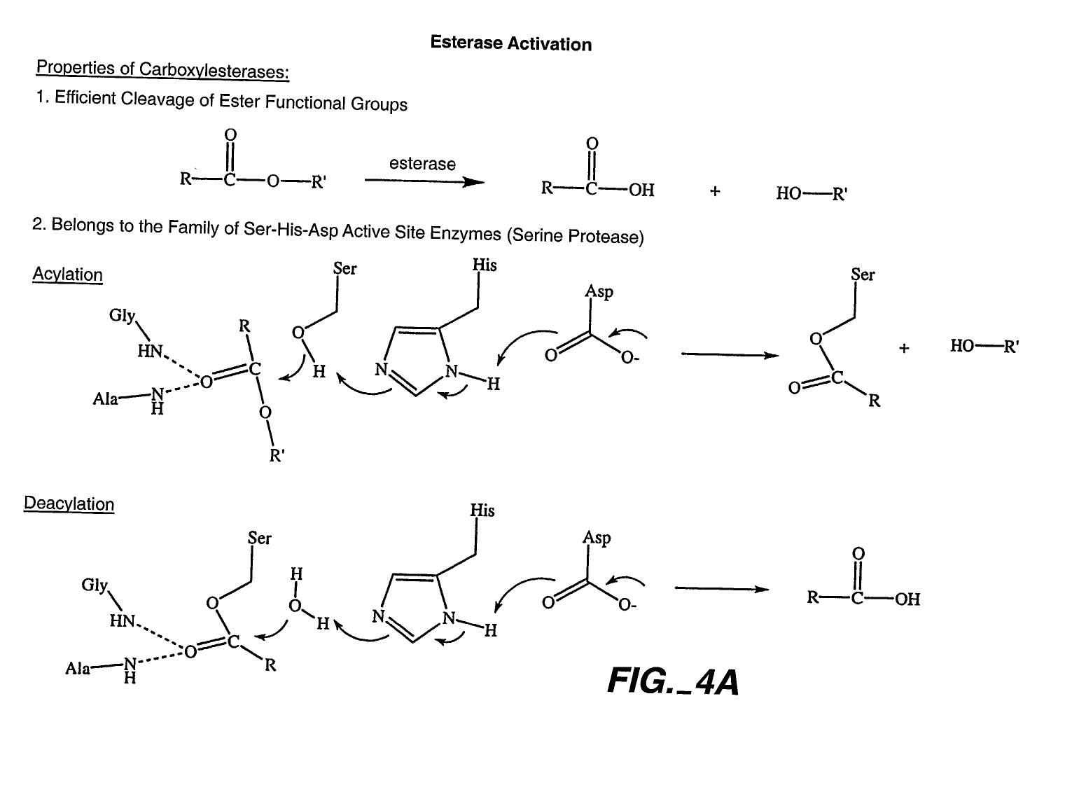

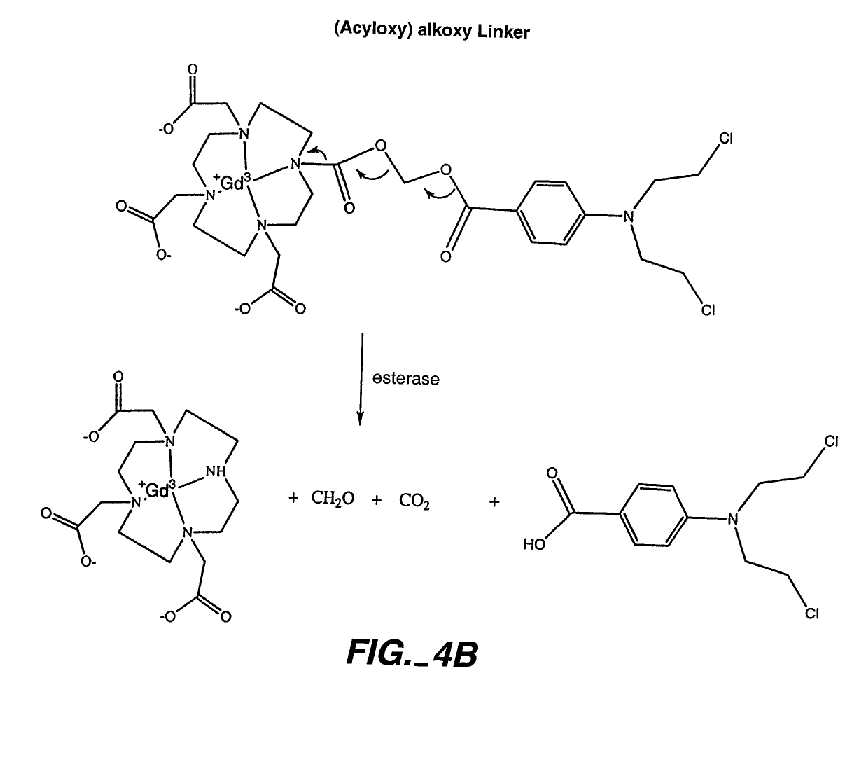

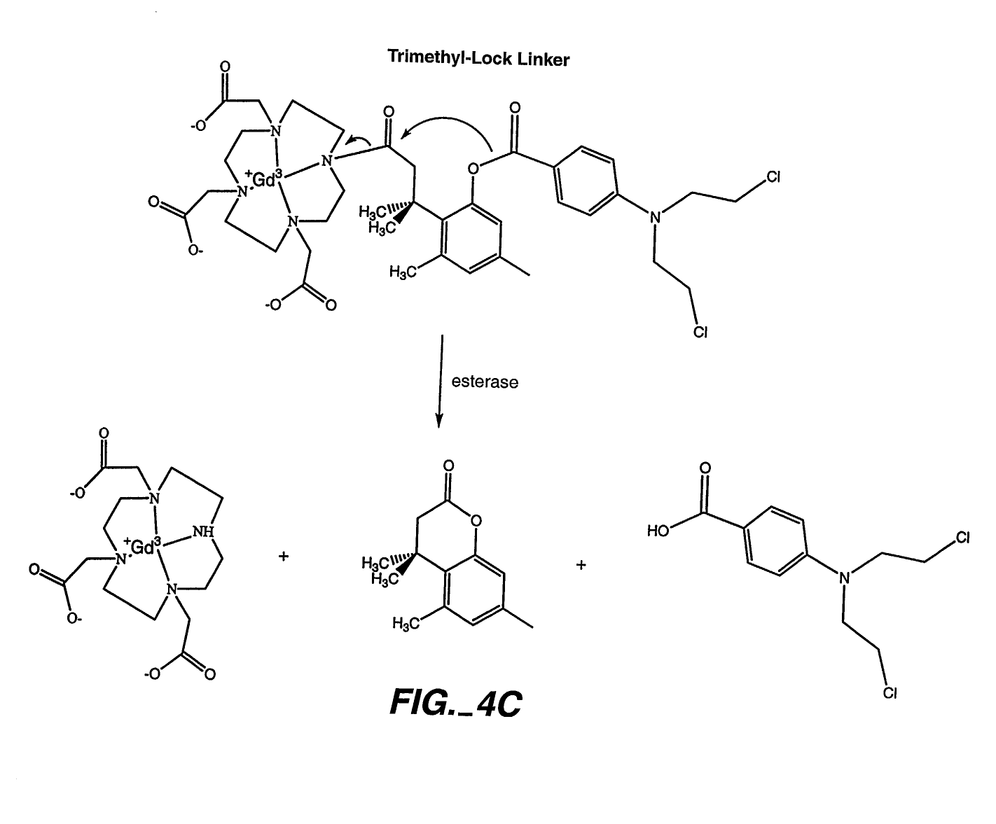

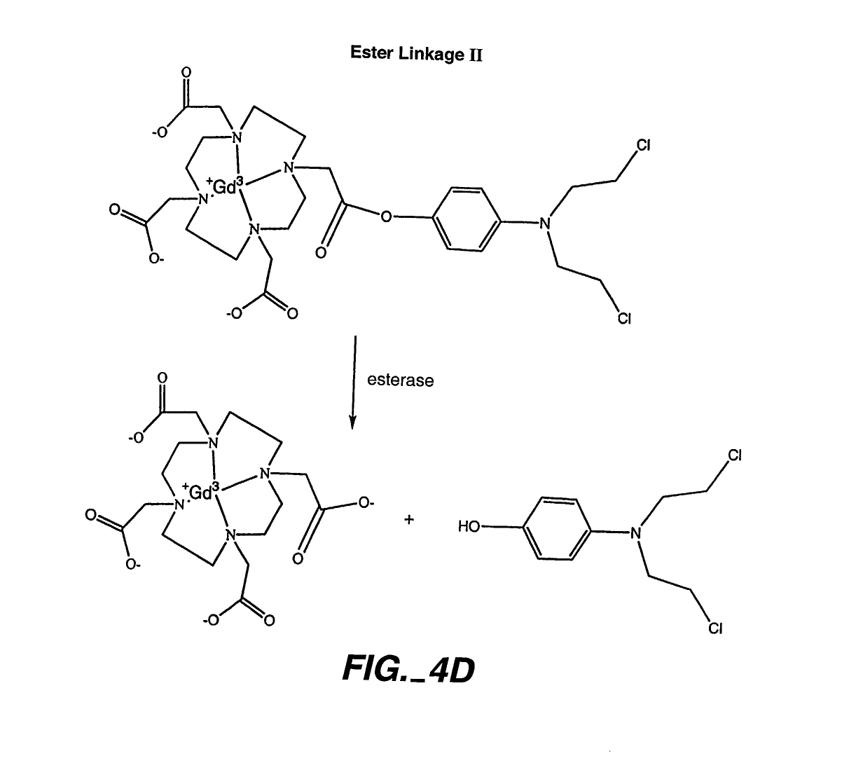

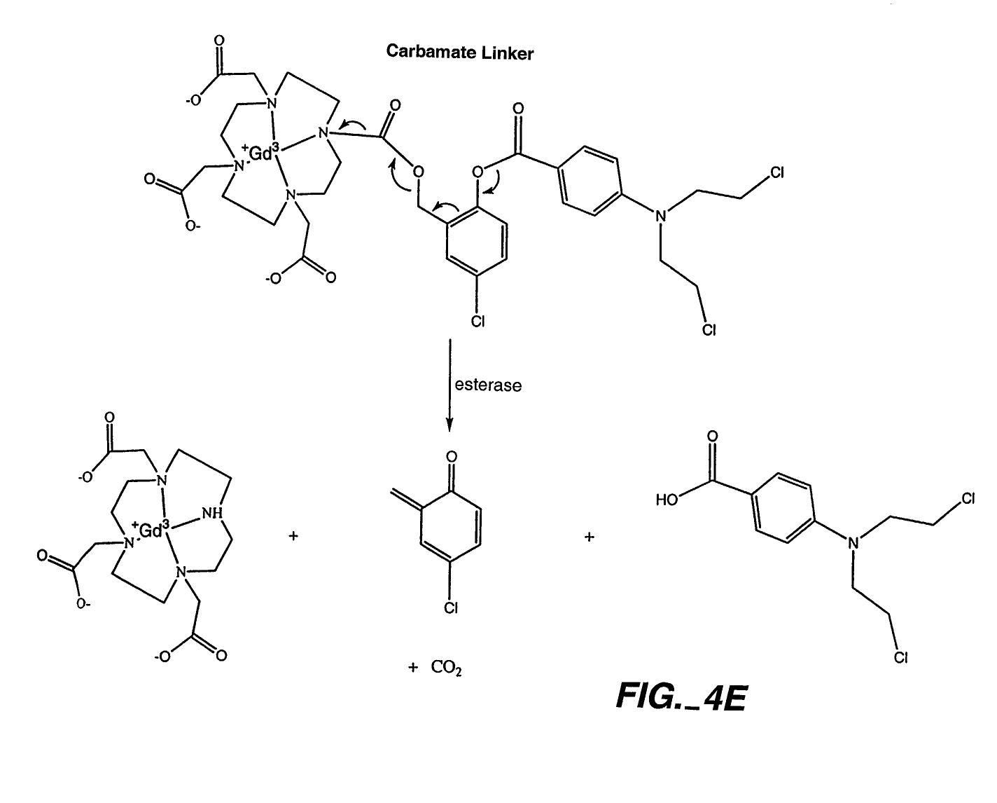

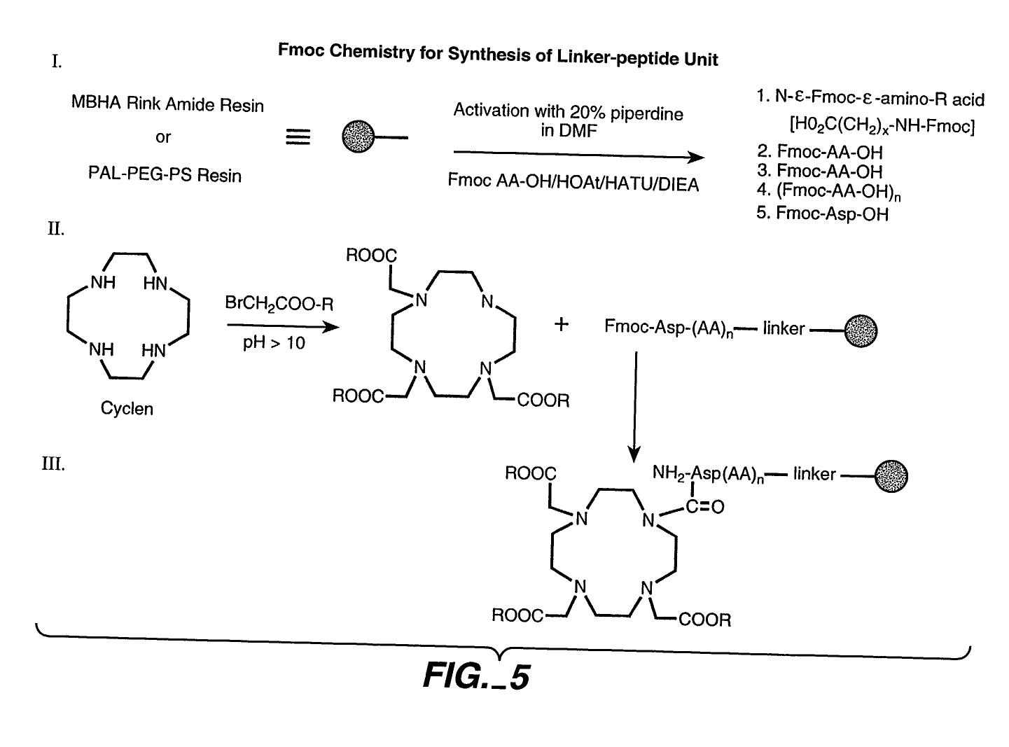

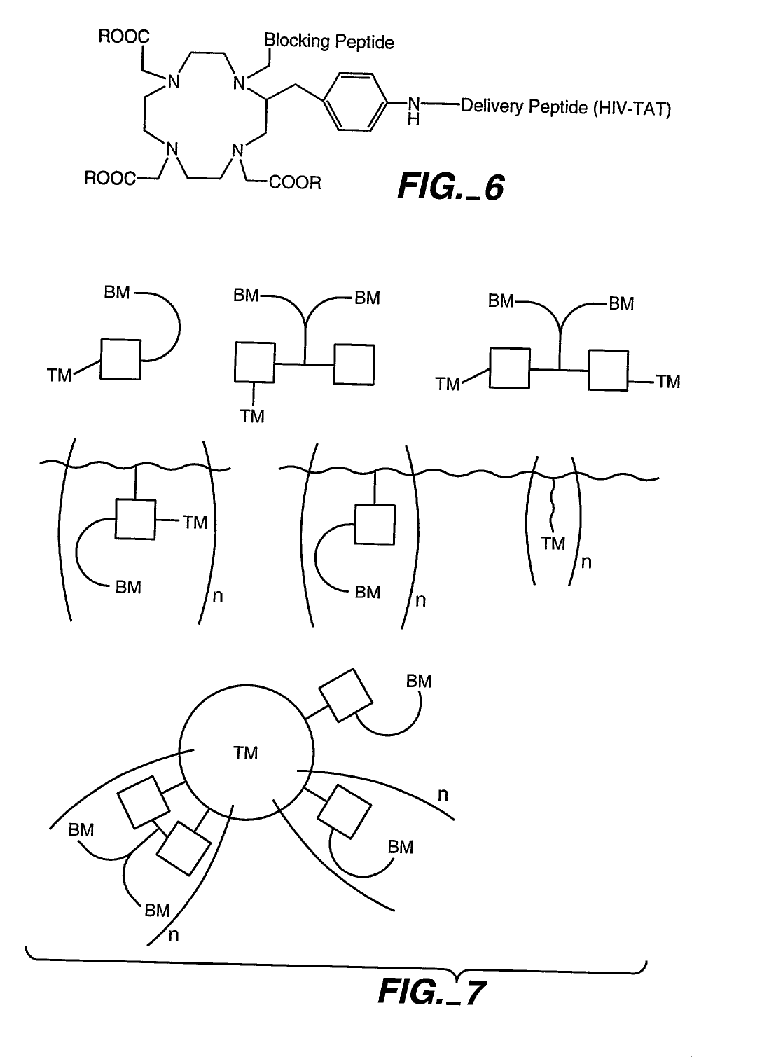

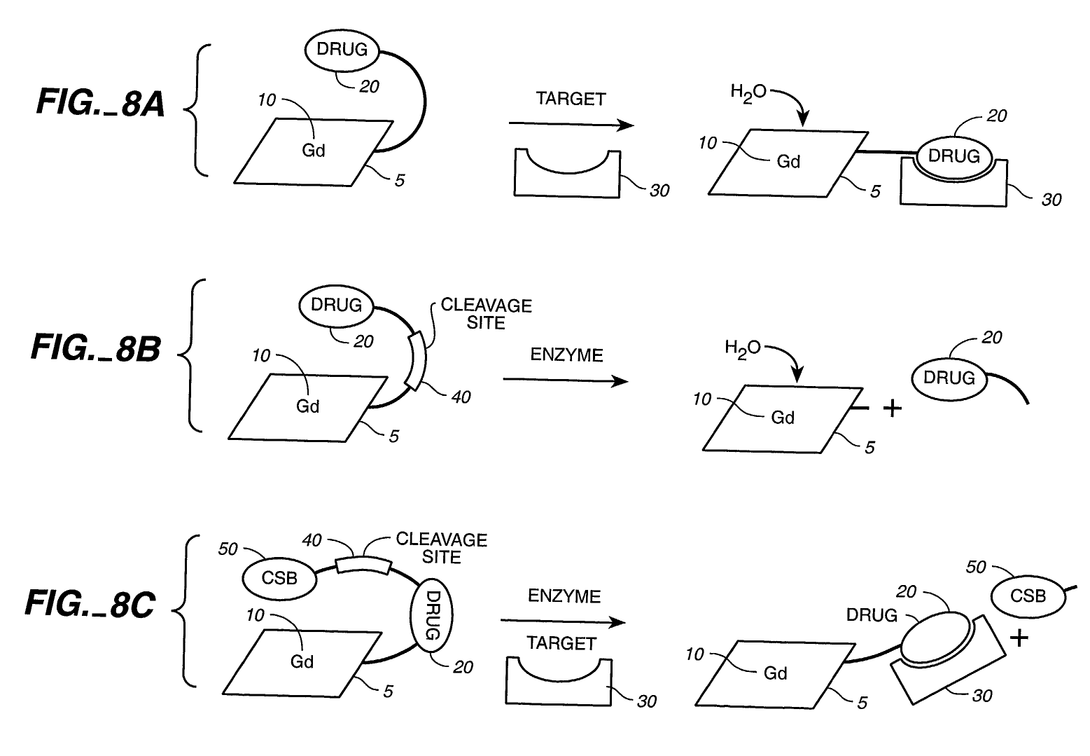

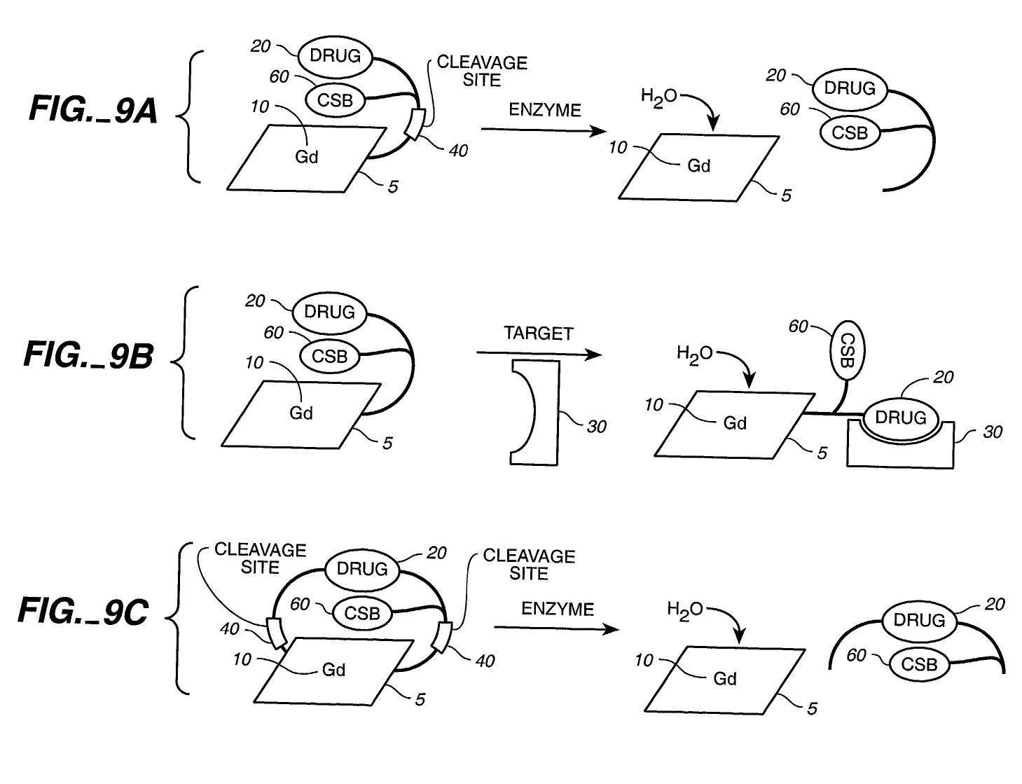

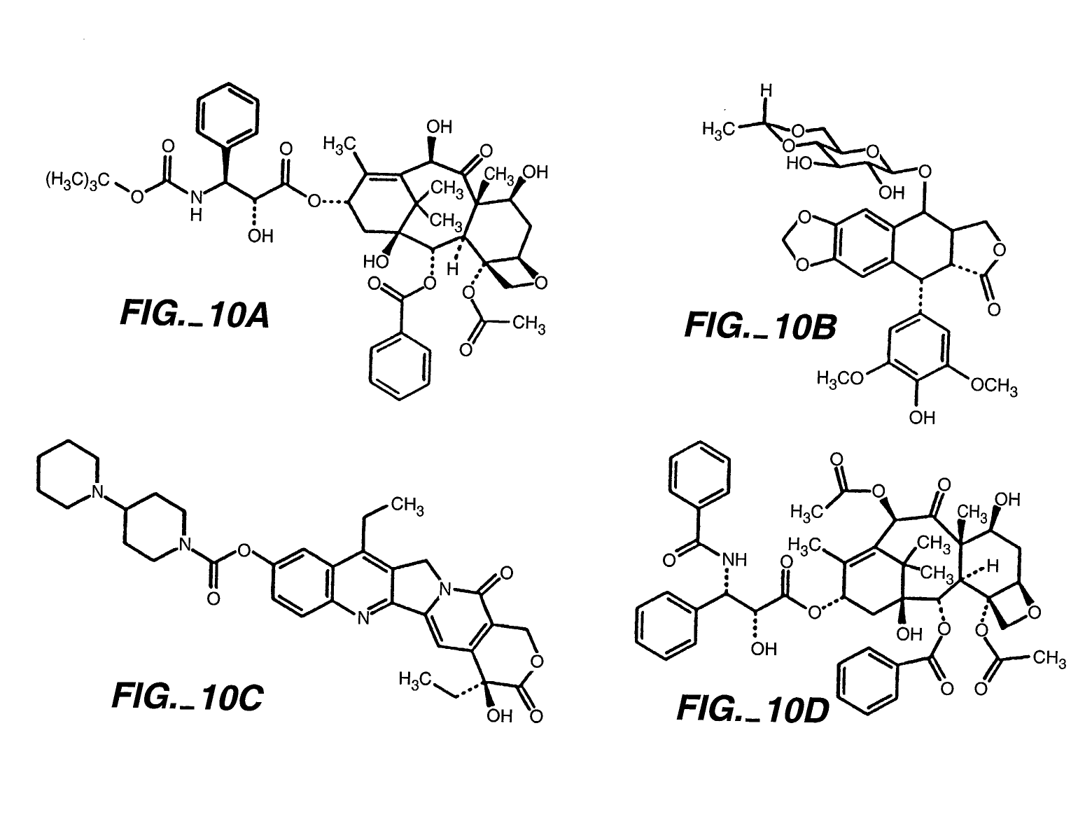

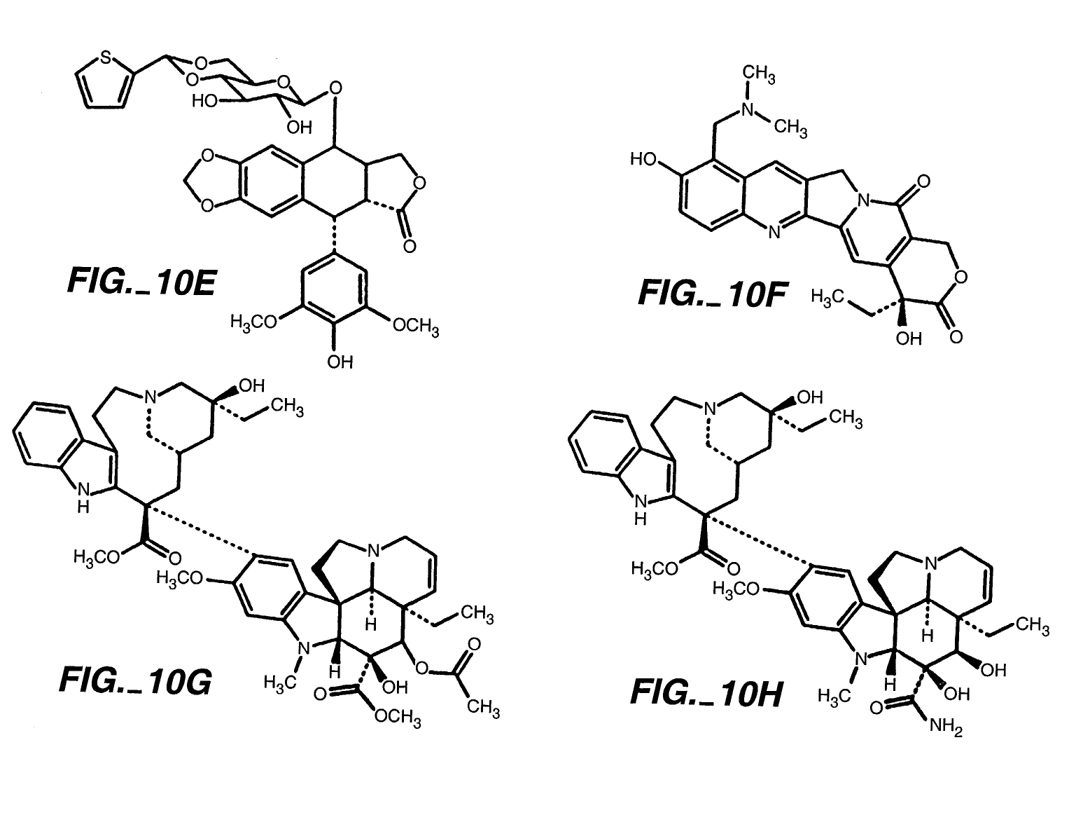

In a preferred embodiment, a coordination site barrier can be attached by a cleavable linker as outlined herein. A preferred embodiment utilizes esterase linkages such as are generally depicted in FIG. 4. Esterase linkages are particularly preferred when the blocking moiety is attached via an “arm” of the chelate, as the product of an esterase reaction is a carboxylic acid, which thus allows the regeneration of a stable chelate (and, in the case of DOTA and DPTA, chelates that are approved for human use). Alternatively, cleavable peptide linkers can also be used.

The blocking moiety is attached to the metal ion complex in a variety of ways. In a preferred embodiment, as noted above, the blocking moiety is attached to the metal ion complex via a linker group. Alternatively, the blocking moiety is attached directly to the metal ion complex; for example, as outlined below, the blocking moiety may be a substituent group on the chelator.

In a preferred embodiment at least one of the R groups attached to the “arms” of the chelator, for example R₉, R₁₀, R₁₁ or R₁₂ of the DOTA structures, or R₁₃, R₁₄, R₁₇, R₂₀ or R₂₁ of the DTPA structures, comprises an alkyl (including substituted and heteroalkyl groups), or aryl (including substituted and heteroaryl groups), i.e. is a group sterically bulkier than hydrogen. This is particular useful to drive the equilibrium towards “locking” the coordination atom of the arm into place to prevent water exchange, as is known for standard MRI contrast agents. Preferred groups include the C1 through C6 alkyl groups with methyl being particularly preferred.

This is particularly preferred when the blocking moiety is attached via one of the “arms”, for example when a blocking moiety is at position X₁ to X₄ (Structure 6), position S, T, U or V (Structure 8) or position H, I, J or K of Structure 16.

However the inclusion of too many groups may drive the equilibrium in the other direction effectively locking the coordination atom out of position. Therefore in a preferred embodiment only 1 or 2 of these positions is a non-hydrogen group, unless other methods are used to drive the equilibrium towards binding.

The blocking moieties are chosen and designed using a variety of parameters. In the embodiment which uses a coordination site barrier, i.e. when the functional group of the blocking moiety does not provide a coordination atom, and the coordination site barrier is fastened or secured on two sides, the affinity of the coordination site barrier of the blocking moiety for the metal ion complex need not be great, since it is tethered in place. That is, in this embodiment, the complex is “off” in the absence of the target substance. However, in the embodiment where the blocking moiety is linked to the complex in such a manner as to allow some rotation or flexibility of the blocking moiety, for example, it is linked on one side only, the blocking moiety should be designed such that it occupies the coordination site a majority of the time. For example, a galactose-DOTA structure in which the galactose is tethered on one side only gives roughly a 20% increase in the signal in the presence of galactosidase, thus indicating that the galactose blocking moiety is in equilibrium between blocking or occupying the coordination site and rotating free in solution. However, as described herein, these agents may be “locked” off using R groups on the carboxylic acid “arms” of a chelator, to reduce the rotational freedom of the group and thus effectively drive the equilibrium to the “off” position, and thus result in a larger percentage increase in the signal in the presence of the target.

When the blocking moiety is not covalently tethered on two sides, as is depicted in FIG. 2, it should be understood that blocking moieties and coordination site barriers are chosen to maximize three basic interactions that allow the blocking moiety to be sufficiently associated with the complex to hinder the rapid exchange of water in at least one coordination site of the complex. First, there may be electrostatic interactions between the blocking moiety and the metal ion, to allow the blocking moiety to associate with the complex. Secondly, there may be Van der Waals and dipole-dipole interactions. Thirdly, there may be ligand interactions, that is, one or more functionalities of the blocking moiety may serve as coordination atoms for the metal. In addition, linker groups may be chosen to force or favor certain conformations, to drive the equilibrium towards an associated blocking moiety. Similarly, removing degrees of fredom in the molecule may force a particular conformation to prevail. Thus, for example, the addition of alkyl groups, and particularly methyl groups, at positions equivalent to the R₉ to R₁₂ positions of Structure 7 when the blocking moiety is attached at W, X, Y or Z, can lead the blocking moiety to favor the blocking position. Similar restrictions can be made in the other embodiments, as will be appreciated by those in the art.

Furthermore, effective “tethering” of the blocking moiety down over the metal ion may also be done by engineering in other non-covalent interactions that will serve to increase the affinity of the blocking moiety to the chelator complex, as is depicted below.

Potential blocking moieties may be easily tested to see if they are functional; that is, if they sufficiently occupy or block the appropriate coordination site or sites of the complex to prevent rapid exchange of water. Thus, for example, complexes are made with potential blocking moieties and then compared with the chelator without the blocking moiety in imaging experiments. Once it is shown that the blocking moiety is a sufficient “blocker”, the target substance is added and the experiments repeated, to show that interaction with the target substance increases the exchange of water and thus enhances the image.

There are a wide variety of suitable activitable agents that may be used in the present invention, many of which are depicted in the Figures, and outlined in U.S. Pat. No. 5,707,605, PCT US96/08549, and U.S. Ser. No. 09/134,072, U.S. Pat. No. 5,980,862, U.S. Ser. No. 09/405,046, No. 09/866,512; No. 09/179,927, No. 09/716,175, PCT/US01/14665; U.S. Ser. No. 09/908,436; No. 09/972,302; U.S.S. No. 60/288,963; U.S.S. No. 60/282,136; U.S. Ser. No. 10/16,706 No. 60/285,602; and a utility application by Meade, entitled “Increasing In-Vivo Residence Time of AMR Contrast Agent”, filed Apr. 22, 2002; all of which are expressly incorporated herein by reference.

In a preferred embodiment, the agents of the invention are not activatible; that is, they are always “on”. These agents are well known in the art and can comprise any number of additional moieties, including the R groups listed herein, including targeting moieties, polymers, etc.

Accordingly, in a preferred embodiment, the present invention provides MRI agents comprising candidate agents. The term “candidate bioactive agent” or “exogeneous compound” as used herein describes any molecule, e.g., protein, oligopeptide, small organic molecule, polysaccharide, polynucleotide, etc. that can be screened for activity as outlined herein. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein.

Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occuring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, as is more fully outlined below, the candidate agents are either randomized proteins (including biased proteins or proteins with fusion partners) or expression products of cDNA libraries or libraries derived from cDNA libraries, such as fragmented (including randomly fragmented cDNA libraries). These are added to the cells as nucleic acids encoding these proteins. As will be appreciated by those in the art, these cDNA libraries may be full length or fragments, and can be in-frame, out-of-frame or read from the anti-sense strand.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

As described above generally for proteins, nucleic acid candidate bioactive agents may be naturally occuring nucleic acids, random nucleic acids, or “biased” random nucleic acids. For example, digests of procaryotic or eucaryotic genomes or cDNA libraries may be used as is outlined above for proteins.

In a preferred embodiment, the candidate bioactive agents are organic chemical moieties, a wide variety of which are available in the literature.

In a preferred embodiment, a library of different candidate bioactive agents are used. Preferably, the library should provide a sufficiently structurally diverse population of randomized agents to effect a probabilistically sufficient range of diversity to allow binding to a particular target. Accordingly, an interaction library should be large enough so that at least one of its members will have a structure that gives it affinity for the target. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, such as generally proposed herein, has the potential to code for 20⁷ (10⁹) to 20²⁰ . Thus, with libraries of 10⁷ to 10⁸ different molecules the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different sequences are simultaneously analyzed in the subject methods. Preferred methods maximize library size and diversity.

The candidate bioactive agents are combined or added to the chelates using known chemistries, as generally outlined in U. S. application Ser. No. 09/179,927 filed Oct. 27, 1998; No. 60/201,817 filed May 4, 2000; No. 60/203, 224 filed May 5, 200; Ser. No. 09/716,175 filed Nov. 16, 2000; Ser. No. 08/460,511; filed Jun. 2, 1995; Ser. No. 08,486,968 filed Jun. 7, 1995; No. 60/063,328 filed Oct. 27, 1997; Ser. No. 08/971,855 filed Nov. 17, 1997; Ser. No. 09/134,072 filed Aug. 13, 1998; Ser. No. 09/405,046 filed Sep. 27, 1999; No. 60/287,619 filed May 26, 2000; No. 60/202,108 filed May 4, 2000; Ser. No. 09/716,178 filed Nov. 16, 2000; No. 60/201,816 filed May 4, 2000; Ser. No. 09/715,859 filed Nov. 17, 2000, all of which are expressly incorporated by reference in their entirety. In addition, linkers may be included, as well as targeting moieties.

By the term “targeting moiety” herein is meant a functional group that serves to target or direct the complex to a particular location or association, i.e. a specific binding event. Thus, for example, a targeting moiety may be used to target a molecule to a specific target protein or enzyme, or to a particular cellular location, or to a particular cell type. Suitable targeting moieties include, but are not limited to, polypeptides, nucleic acids, carbohydrates, lipids, hormones including proteinaceous and steroid hormones, growth factors, receptor ligands, antigens and antibodies, and the like. For example, as is more fully outlined below, a therapeutically active agent such as the cobalt compounds outlined below may include a targeting moiety to specifically bind a particular protein. Alternatively, as is more fully outlined below, the MRI agents of the invention may include a targeting moiety to target the agents to a specific cell type such as tumor cells, such as a transferrin moiety, since many tumor cells have significant transferrin receptors on their surfaces. Similarly, a targeting moiety may include components useful in targeting the MRI agents or the therapeutically active agents (if released) to a particular subcellular location. As will be appreciated by those in the art, the localization of proteins within a cell is a simple method for increasing effective concentration. For example, shuttling a drug into the nucleus confines them to a smaller space thereby increasing concentration. Finally, the physiological target may simply be localized to a specific compartment, and the drugs must be localized appropriately.

Thus, suitable targeting sequences include, but are not limited to, binding sequences capable of causing binding of the moiety to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations.

In some embodiments, the targeting moiety replaces a coordination atom, although this is not generally preferred. By “targeting moiety” herein is meant a functional group which serves to target or direct the complex to a particular location, cell type, diseased tissue, or association. In general, the targeting moiety is directed against a target molecule. As will be appreciated by those in the art, the MRI contrast agents of the invention are generally injected intraveneously; thus preferred targeting moieties are those that allow concentration of the agents in a particular localization. Thus, for example, antibodies, cell surface receptor ligands and hormones, lipids, sugars and dextrans, alcohols, bile acids, fatty acids, amino acids, peptides and nucleic acids may all be attached to localize or target the contrast agent to a particular site.

In a preferred embodiment, the targeting moiety allows targeting of the MRI agents of the invention to a particular tissue or the surface of a cell. That is, in a preferred embodiment the MRI agents of the invention need not be taken up into the cytoplasm of a cell to be activated.

In a preferred embodiment, the targeting moiety is a peptide. For example, chemotactic peptides have been used to image tissue injury and inflammation, particularly by bacterial infection; see WO 97/14443, hereby expressly incorporated by reference in its entirety.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells, including, but not limited to, HER2, VEGF, etc.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In one embodiment, antibodies against virus or bacteria can be used as targeting moieties. As will be appreciated by those in the art, antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like) may be used.

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

In a preferred embodiment, the targeting moiety is a carbohydrate. By “carbohydrate” herein is meant a compound with the general formula Cx(H2O)y. Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, man nose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety.

In a preferred embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In addition, as will be appreciated by those in the art, any moiety which may be utilized as a blocking moiety can be used as a targeting moiety. Particularly preferred in this regard are enzyme inhibitors, as they will not be cleaved off and will serve to localize the MRI agent in the location of the enzyme.

In a preferred embodiment, the targeting moiety may be used to either allow the internalization of the MRI agent to the cell cytoplasm or localize it to a particular cellular compartment, such as the nucleus.

In a preferred embodiment, the targeting moiety is all or a portion of the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In a preferred embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799,1987; Galileo, et al., Proc. Natl. Acad. Sci. USA, 87:458-462,1990.

In a preferred embodiment, targeting moieties for the hepatobiliary system are used; see U.S. Pat. Nos. 5,573,752 and 5,582,814, both of which are hereby incorporated by reference in their entirety.

Another class of suitable substitution groups are chemical functional groups that are used to add the components of the invention together, as is more fully outlined below. Thus, in general, the components of the invention are attached through the use of functional groups on each that can then be used for attachment. Preferred functional groups for attachment are amino groups, carboxy groups, oxo groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, incorporated herein by reference). Preferred linkers include, but are not limited to, alkyl groups (including substituted alkyl groups and alkyl groups containing heteroatom moieties), with short alkyl groups, esters, amide, amine, epoxy groups, nucleic acids, peptides and ethylene glycol and derivatives being preferred.

Once made, the compositions of the invention, including the libraries of MRI contrast agents, may be used in a wide variety of applications. The agents of the invention may be used in a similar manner to the known gadolinium MRI agents. See for example, Meyer et al., supra; U.S. Pat. No. 5,155,215; U.S. Pat. No. 5,087,440; Margerstadt et al., Magn. Reson. Med. 3:808 (1986); Runge et al., Radiology 166:835 (1988); and Bousquet et al., Radiology 166:693 (1988). The metal ion complexes are administered to a cell, tissue or patient as is known in the art. A “patient” for the purposes of the present invention includes both humans and other animals and organisms, such as experimental animals. Thus the methods are applicable to both human therapy and veterinary applications. In addition, the metal ion complexes of the invention may be used to image tissues or cells; for example, see Aguayo et al., Nature 322:190 (1986).

Generally, sterile aqueous solutions of the contrast agent complexes of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred. Dosages may depend on the structures to be imaged. Suitable dosage levels for similar complexes are outlined in U.S. Pat. Nos. 4,885,363 and 5,358,704.

In addition, the contrast agents of the invention may be delivered via specialized delivery systems, for example, within liposomes (see Navon, Magn. Reson. Med. 3:876-880 (1986)) or microspheres, which may be selectively taken up by different organs (see U.S. Pat. No. 5,155,215).

In some embodiments, it may be desirable to increase the blood clearance times (or half-life) of the MRI agents of the invention. This has been done, for example, by adding carbohydrate polymers to the chelator (see U.S. Pat. No. 5,155,215). Thus, one embodiment utilizes polysaccharides as substitution R groups on the compositions of the invention.

A preferred embodiment utilizes complexes which cross the blood-brain barrier. Thus, as is known in the art, a DOTA derivative which has one of the carboxylic acids replaced by an alcohol to form a neutral DOTA derivative has been shown to cross the blood-brain barrier. Thus, for example, neutral complexes are designed that cross the blood-brain barrier with blocking moieties which detect Ca+2 ions. These compounds are used in MRI of a variety of neurological disorders, including Alzeheimer's disease. Currently it is difficult to correctly diagnosis Alzeheimer's disease, and it would be useful to be able to have a physiological basis to distinguish Alzeheimer's disease from depression, or other treatable clinical symptoms for example. In addition, compounds including, but are not limited to, congo red, BSB, and chrysamine G can be used as amyloid binding moieties (ABM) that are known to cross the blood brain barrier and bind to amyloid plaques; see U.S. Ser. No. 09/972,302, hereby expressly incorporated by reference in its entirety.

In a preferred embodiment, the compositions of the invention are used in gene and/or protein expression analysis. The emerging importance of genomic and proteomic information is rapidly altering the way pharmaceutical research is conducted. The use of molecular biology tools in the discovery process is important to many drug discovery efforts. Among the most used tools in this context are gene expression reporter systems. In cultured cells, these technologies are used to assess gene induction/suppression. The imaging agents of the invention can also be used in this fashion and provide a significant advantage over the technologies currently on the market.

The most classical technique used to assess in vivo gene expression is to introduce a gene to the genome of a test animal. These introduced genes (transgenes) are engineered to contain promoters which are constitutively or inducibly active in the tissue of choice placed upstream from the coding sequence of the gene product. A second sequence coding for an easily assayed or visualized protein (reporter gene) is included immediately downstream from the transgene. The transcription of both the transgene and reporter gene is driven by the same promoter, therefore expression occurs in the same tissues. Assessment of the expression of the routine and more easily measured reporter is used to verify the tissue-specific and/or drug influenced expression of the transgene. Changes in the expression of the reporter gene results from treatment of the cells with candidate compounds which influence gene expression. This approach will continue to play a significant role in pharmaceutical discovery and development over the next several years. MetaProbe has already been designed and demonstrated as a tool to measure gene expression.

Often pharmaceuticals are sought which alter the expression of a particular gene. Candidate compounds are screened for influence on the transcription of that gene. To do so, the gene's promoter is inserted upstream from the coding sequence of a reporter protein.

Accordingly, the compositions of the invention are used in gene and/or protein expression analysis. As outlined herein, this may be done in a variety of ways. As outlined above, in one embodiment, an MRI agent, either activatible by the presence of the gene product or protein or that will bind to the gene product or protein using a suitable targeting moiety (e.g. binding partner of the gene product or protein) is used to follow gene expression. That is, an activatible agent is administered to the animal or cell and the presence of the target gene product or protein is monitored. This can be repeated over time, depending on growth conditions, the presence or absence of a disease state, normal development (for example, in utero embryonic development), drug administration, etc. The ability to do non-invasive studies of the same animal over time is a significant benefit.

Alternatively, the target gene can be hooked to a gene encoding a reporter protein that will bind and/or be activated by the MRI contrast agent, as discussed above.

IN addition, the compositions of the invention find use in target analyte identification. That is, knowledge that a particular gene is specifically involved in a given biological process makes the protein encoded by that gene an excellent target for manipulation by small molecule drugs. In addition, one can envision therapies involving the delivery of that protein itself. However, while knowledge of gene sequence increases the number of targets available, it dose not inherently make target identification easier. In fact, the abundance of new genomic data has meant a proliferation of potential “target” genes without a corresponding increase in knowledge on the basis of which to differentiate them. Although raw sequence data can be “annotated” by making comparisons between uncharacterized sequences and those of known gene families, the relatively general relationships these analyses produce still necessitate a great deal of further work to establish any functional role or disease relevance for a particular gene. With the completed sequence of the genome having dramatically increased the inflow of potential targets into drug discovery programs, the critical next requirement is to develop methods to efficiently validate those targets.

The completion of the human genome is expected to dramatically expand drug discovery horizons. It has been estimated that 5,000 or more new drug discovery targets will emerge over the next several years from the thousands of genes that have been newly uncovered. A decade ago, fewer than 10% of human genes were known, which necessarily severely restricted drug discovery possibilities. In fact, it has been estimated that all of the prescription drugs now available attack a mere 500 targets.

In reality, the human genome has been revealed to be much smaller than was widely anticipated: 30,000 to 40,000 genes versus earlier estimates of 100,000 or more. However, this does not imply that drug discovery possibilities are much more limited than was previously believed. It is apparent that much of the complexity of human biology lay not in simple gene number, but rather in gene product number. One gene, as it turns out, makes more than one protein product, contrary to old biological dogma. As such, 30,000 genes could well translate into 250,000 or more distinct proteins, many of which may constitute viable drug discovery targets. In addition, protein-protein interactions constitute another potential set of drug discovery targets. The total potential number of new drug discovery targets is not limited to gene number, but actually should be some multiple of that figure.

Similarly, Functional genomics, the study of gene function in cells and organisms, has elucidated many previously unknown biochemical pathways, which has exponentially increased the number of drug targets. For example, each complex cellular process uses several enzymes to perform individual steps of the process. Therefore, an error in any one of the enzymes can affect the outcome of the process, potentially causing disease. However, an upstream or downstream molecule in the same process may still make a good drug discovery target. By increasing the knowledge of the cellular basis of observed diseases, functional genomics has been integral in determining the types of targets most useful for drug discovery.

One of the most important uses of functional genomics is target validation, or the process of identifying which proteins are candidates for drug discovery. In fact, as genomics has successfully identified potential drug targets, the industry's bottleneck has moved from target identification to validation.

Thus, as outlined herein, agents that will bind to various targets can be tested as outlined herein.

In a preferred embodiment, the libraries of the agents of the invention may be screened in a variety of ways, for a variety of reasons.

In a preferred embodiment, the MRI agents are screened simply for their ability to bind to a target analyte. That is, given a protein of interest in a disease state (e.g. a target protein), a drug, etc., methods are provided to screen libraries of MRI agents, each comprising a different candidate agent such as a peptide, for binding to the target analyte. IN a preferred embodiment, the binding assays are done by providing a substrate with an immobilized target analyte. By “substrate” or “solid support” or other grammatical equivalents herein is meant any material that can be modified to contain discrete individual sites appropriate of the attachment or association of capture ligands. Suitable substrates include metal surfaces such as gold, electrodes as defined below, glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses and a variety of other polymers. Microtiter plates are particularly preferred.

Thus, generally the target analytes are immobilized and the MRI agents added, although this may be reversed. This is generally done at physiological conditions, with physiologically acceptable buffers. Washing and incubation steps can be done, followed by detection of binding. This also can be done in a variety of ways. In a preferred embodiment, this is done through magnetic resonance imaging, as is well known in the art. However, in some cases, fluorescence-conferring metal ions, rather than paramagnetic metal ions are used. Thus Europium and Terbium, as well as other metals known to confer fluorescence in the chelated state, may be used for imaging.

In addition, the library of agents may be tested for “activatibility”. In this embodiment, the agents are first subjected to magnetic resonance imaging, followed by exposure to target analytes or candidate target analytes, and then a second image taken. Those agents that show an increase in contrast are “activatible”. Thus, for example, libraries of agents comprising different peptides, whether random, biased random, cDNA fragments (either in frame, out of frame or from antisense coding, as outlined above) are added to chelates comprising paramagnetic metal ions. The library is exposed to one or more proteases and images acquired. For example, if agents activated by specific proteases are required, specific proteases may be used. Alternatively, proteases with broad specificity can be used to generate activatible libraries that then can be secondarily screened for specificity with other proteases or within animals.

In a preferred embodiment, the agents of the invention (again, either activatible or non-activatible) can be used in time course studies for a variety of reasons. For example, time-dependent studies of development, sexual maturation, aging, disease progression, response to drugs or candidate drugs, etc., can all be run. In general, these methods comprise administering an agent of the invention to an animal, tissue or cell, taking an image, and then repeating the process one or more times over time.

In a preferred embodiment, time experiments are done with two or more agents of the invention. In a preferred embodiment, agents are administered (either preferably sequentially or in some cases, simultaneously) to animals, tissues or cells and images taken. Thus, for example, agents that bind to different proteins within a single signalling pathway, or to proteins within different (e.g. some related) signalling pathways may be administered sequentially and/or over time as well.

In addition, the present invention can utilize robotic systems. In a preferred embodiment, the devices of the invention comprise liquid handling components, including components for loading and unloading fluids at each station or sets of stations. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; holders with cartridges and/or caps; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.; this is in addition to or in place of the station thermocontrollers.

In a preferred embodiment, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components

The agents of the invention are administered to a cell, tissue or patient as is known in the art. A “patient” for the purposes of the present invention includes both humans and other animals and organisms, such as experimental animals. Thus the methods are applicable to both human therapy and veterinary applications. In addition, the metal ion complexes of the invention may be used to image tissues or cells; for example, see Aguayo et al., Nature 322:190 (1986).

Generally, sterile aqueous solutions of the contrast agent complexes of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred. Dosages may depend on the structures to be imaged. Suitable dosage levels for similar complexes are outlined in U.S. Pat. Nos. 4,885,363 and 5,358,704.

In addition, the contrast agents of the invention may be delivered via specialized delivery systems, for example, within liposomes (see Navon, Magn. Reson. Med. 3:876-880 (1986)) or microspheres, which may be selectively taken up by different organs (see U.S. Pat. No. 5,155,215).

In some embodiments, it may be desirable to increase the blood clearance times (or half-life) of the MRI agents of the invention. This has been done, for example, by adding carbohydrate polymers to the chelator (see U.S. Pat. No. 5,155,215). Thus, one embodiment utilizes polysaccharides as substitution R groups on the compositions of the invention.

A preferred embodiment utilizes complexes which cross the blood-brain barrier. Thus, as is known in the art, a DOTA derivative which has one of the carboxylic acids replaced by an alcohol to form a neutral DOTA derivative has been shown to cross the blood-brain barrier. Thus, for example, neutral complexes are designed that cross the blood-brain barrier with therapeutic blocking moieties to treat disorders of the brain.

In a preferred embodiment, the therapeutic moiety is attached to the chelate using a photocleavable moiety as defined on the next page. Also included in the photocleavable moieties are guinone derivatives. Also included are WO99/25389, PCT US/9822743, hereby incorporated by reference.

The references cited herein are expressly incorporated by reference in their entirety.

1. A library of MRI contrast agents each comprising:
a) a chelate;
b) a paramagnetic metal ion; and
c) a different candidate agent covalently attached to said chelate.

2. A library according to claim 1 wherein said MRI contrast agents each comprise:
a) said paramagnetic metal ion capable of binding n coordination atoms, wherein said metal ion is bound to said chelator such that said metal ion has coordination atoms at (n-1) or (n-2) coordination sites of said metal ion; and
b) a blocking moiety covalently attached to said chelator which hinders the rapid exchange of water in the remaining coordination site or sites;
wherein said blocking moiety is capable of interacting with a target substance such that the exchange of water in said remaining coordination sites is increased.

3. A library according to claim 1 wherein said candidate agents are peptides.

4. A library according to claim 2 wherein said peptides comprise fully random peptides.

5. A library according to claim 2 wherein said peptides comprise partially random peptides.

6. A library according to claim 1 wherein said candidate agents are carbohydrates.

7. A library according to claim 1 wherein said candidate agents are nucleic acids.

8. A library according to claim 1 wherein said candidate agents are lipids.

9. A library according to claim 1 wherein said candidate agents are attached to said chelates using a linker.

10. A library according to claim 5 wherein said linker is photocleavable.

11. A library according to claim 1 wherein each contrast agent further comprises a targeting moiety.

12. A library according to claim 1 wherein said library comprises at least 10³ different members.

13. A method of screening for binding interactions comprising:
a) providing a substrate with an immobilized target analyte;
b) contacting said substrate with a library of agents each comprising:
i) a chelate;
ii) a metal ion; and
iii) a different peptide covalently attached to said chelate;
wherein said contacting is done under conditions that allow at least one of said agents to bind to said target analyte;
c) rinsing said substrate to remove unbound agents; and
d) detecting the presence of binding of of at least one of said agents to said target analyte.

14. A method according to claim 13 wherein said metal ion is a paramagnetic metal ion, said agent is an MRI contrast agent and said detecting is done by taking a magnetic resonance image of said substrate.

15. A method according to claim 13 wherein said metal ion confers fluorescence to said agent and said detecting is done by detecting fluorescence on said substrate.

16. A method according to claim 14 or 15 wherein said substrate comprises a plurality of different target analytes.

17. A method according to claim 14 or 15 wherein said method utilizes a robotic system.

18. A method of screening for protease-activated MRI contrast agents comprising:
a) providing a substrate with a plurality of test sites, each test site comprising at least one MRI contrast agent comprising a paramagnetic metal ion bound to a complex, said complex comprising:
i) a chelator; and
ii) a peptide blocking moiety covalently attached to said chelator which binds in at least a first coordination site of said metal ion such that the exchange of water in said first coordination site is hindered;
wherein at least two of said test sites comprising a different peptide blocking moiety;
b) adding a first protease to each of said test sites;
c) taking a Magnetic Resonance Image of said substrate, wherein if an increase in image intensity is observed, said test site comprises a protease-activated MRI contrast agent.

19. A method according to claim 18 wherein each of said test sites comprises a single MRI contrast agent.

20. A method according to claim 18 wherein each of said test sites comprises a plurality of MRI contrast agents.

21. A method according to claim 18 further comprising identifying the activated MRI contrast agent.

22. A method according to claim 18 wherein said method utilizes a robotic system.

23. A method of making a library of MRI contrast agents comprising:
a) providing a chelate with a first functional group;
b) providing a library of candidate agents each with a second functional group;
c) covalently attaching said first and second functional groups.

24. A method according to claim 23 wherein said candidate agents are peptides.

25. A method according to claim 23 wherein said method utilizes a robotic system.

26. A method of imaging an animal comprising:
a) injecting said animal with a first MRI contrast agent comprising a paramagnetic metal ion bound to a complex, said complex comprising:
i) a chelator; and
ii) a first blocking moiety covalently attached to said chelator which binds in at least a first coordination site of said metal ion and which interacts with a first target substance such that the exchange of water in at least said first coordination site is increased;
b) taking a first Magnetic Resonance Image;
c) injecting said animal with a second MRI contrast agent comprising a paramagnetic metal ion bound to a complex, said complex comprising:
i) a chelator; and
ii) a second blocking moiety covalently attached to said chelator which binds in at least a first coordination site of said metal ion and which interacts with a second target substance such that the exchange of water in at least said first coordination site is increased; and
d) taking a second Magnetic Resonance Image.

27. A method according to claim 26 wherein said animal has been pretreated with a drug candidate before imaging.

28. A method according to claim 26 wherein said animal is a transgenic animal.

29. A method of imaging gene expression of a target gene in an animal comprising:
a) at a first time point, injecting said animal with a MRI contrast agent comprising:
i) a chelate;
ii) a paramagnetic metal ion; and
iii) a moiety comprising a binding partner of the target protein of said target gene, wherein said moiety is covalently attached to said chelate;
b) taking a first magnetic resonance image of said animal;
c) at a second time point, injecting said animal with said MRI contrast agent; and
d) taking a second magnetic resonance image of said animal; and
e) comparing said first and second images to determine the course of gene expression.

30. A method of imaging disease progression in an animal comprising:
a) at a first time point, injecting said animal with a MRI contrast agent comprising:
i) a chelate;
ii) a paramagnetic metal ion; and
iii) a moiety comprising a binding partner of a target protein correlated to said disease, wherein said moiety is covalently attached to said chelate;
b) taking a first magnetic resonance image of said animal;
c) at a second time point, injecting said animal with said MRI contrast agent; and
d) taking a second magnetic resonance image of said animal; and
e) comparing said first and second images to determine the course of said disease.

31. A method according to claim 29 or 30 wherein prior to said second time point, said animal is treated with a drug.

32. A method according to claim 29 or 30 comprising taking repeated images over time.

33. A method according to claim 26, 29 or 30 further comprising sacrificing said animal and performing histological or pathological analyses.

34. A method according to claim 29 or 30 wherein said animal is a transgenic animal.
